(19) United States
(12) Patent Application Publication
(10) Pub. No.: US 2023/0144446 A1
(43) Pub. Date: May 11, 2023

(54) BLOOD-BASED DIAGNOSTIC ASSAYS FOR ALZHEIMER'S DISEASE

(71) Applicant: Cassava Sciences, Inc., Austin, TX (US)

(72) Inventors: Ian Pike, Austin, TX (US); Michael Bremang, Austin, TX (US); George Thornton, Austin, TX (US)

(21) Appl. No.: 17/814,486

(22) Filed: Jul. 22, 2022

Related U.S. Application Data
(60) Provisional application No. 63/225,423, filed on Jul. 23, 2021.

(51) Int. Cl. G01N 33/68

(52) U.S. Cl. CPC G01N 33/6896; G01N 2800/52; G01N 2800/56; G01N 2800/2821

(57) ABSTRACT

The invention relates to sets of biomarkers and methods of use thereof for diagnosing, staging, treating, and assessing the response of a treatment for neurocognitive disorders characterised by tau toxicity, such as Alzheimer’s disease.

### CROSS-REFERENCE TO RELATED APPLICATION

This application claims the benefit of priority to U.S. Provisional Pat. Application No. 63/225,423, filed on Jul. 23, 2021, the entire contents of which is incorporated herein by reference in its entirety.

### REFERENCE TO SEQUENCE LISTING SUBMITTED ELECTRONICALLY

The specification further incorporates by reference a concurrently-filed sequence listing submitted electronically via EFS-Web as a file named “SequenceListing.xml”, created on Jul. 22, 2022. The sequence listing contained in this document is part of the specification and is herein incorporated by reference in its entirety.

### TECHNICAL FIELD

The invention relates to sets of biomarkers and methods of use thereof for diagnosing, staging, treating, and assessing the response of a treatment for neurocognitive disorders characterised by tau toxicity, such as Alzheimer’s disease.

### BACKGROUND

Alzheimer’s disease (AD) represents one of the greatest health care burdens, with 35 million affected individuals worldwide, a population estimated to increase to 115 million by 2050. [Wimo, Alzheimer’s Disease International World Report 2010. The Global Economic Impact of Dementia, Alzheimer’s Disease International (2010).] AD is a devastating dementia that first presents as progressive memory loss and later can include neuropsychiatric symptoms such as depression, paranoia, agitation and even aggression. Currently, available AD treatment is limited to cognitive enhancers with limited and short-lived efficacy.

Previously, diagnosis of AD could only be confirmed at autopsy by the presence of amyloid deposits and neurofibrillary tangles (NFTs) containing the microtubule-associated protein tau. Current clinical diagnoses of AD satisfy the DSM-IV TR and the NINCDS-ADRDA Work Group criteria for probable AD in McKhann et al., Neurology 34(7):939-944 (1984). Initial diagnostic criteria based mostly on subjective assessments set out in McKhann et al., above, require that the presence of cognitive impairment and a suspected dementia syndrome be confirmed by neuropsychological testing for a clinical diagnosis of possible or probable AD; although they need histopathologic confirmation (microscopic examination of brain tissue) for the definitive diagnosis.

The criteria specify eight cognitive domains that can be impaired in AD. Those cognitive domains are: memory, language, perceptual skills, attention, constructive abilities, orientation, problem solving and functional abilities. There are no motor, sensory, or coordination deficits early in the disease. These criteria have shown good reliability and validity, and are those used herein as the basis for assertion of clinical diagnosis of AD.

The diagnosis could not heretofore be determined by laboratory assays. Such assays are important primarily in identifying other possible causes of dementia that should be excluded before the diagnosis of Alzheimer’s disease can be made with confidence. Neuropsychological tests provide confirmatory evidence of the diagnosis of dementia and help to assess the course and response to therapy. The criteria proposed by McKhann et al., above, are intended to serve as a guide for the diagnosis of probable, possible, and definite Alzheimer’s disease; these criteria will likely be revised as more definitive information become available.

Diagnostic criteria have more recently been refined to include the prodromal phase (early symptoms that occur before the full-blown symptoms of the disease are manifest) termed “Mild Cognitive Impairment (MCI) due to AD.” This new diagnosis reflects a desire to treat the disease earlier because the neuropathology is estimated to start 10 years prior to appearance of symptoms. [Trojanowski et al., Alzheimers Dement 6, 230-238 (2010)]. Clinical trials of potential disease-modifying treatments have been hugely disappointing, possibly in part because even an “early-stage” patient already has a massive amyloid-beta (Aβ) burden and substantial pathologies with significant synaptic defects and inflammation.

According to Petersen et al., Arch Neurol 56(3):303-308 (1999), the primary distinction between control subjects and subjects with MCI is in the area of memory, whereas other cognitive functions are comparable. However, when the subjects with MCI were compared with the patients with very mild AD, memory performance was similar, but patients with AD were more impaired in other cognitive domains as well. Longitudinal performance demonstrated that the subjects with MCI declined at a rate greater than that of the controls but less rapidly than the patients with mild AD.

Patients that meet the criteria for MCI can be differentiated from healthy control subjects and those with very mild AD. They appear to constitute a clinical entity that can be characteri zed for treatment interventions.

Amyloid-beta (Aβ) is a peptide 39-42 amino acid residues in length that is generated in vivo by specific, proteolytic cleavage of the amyloid precursor protein (APP) by β- and γsecretases. Aβ₄₂ comprises residues 677-713 of the APP protein, which is itself a 770-residue transmembrane protein having the designation P05067 in the UniProtKB/Swiss-Prot system. Aβ, and in particular the Aβ₄₂, is commonly believed to be the principal causative agent in AD, although its mechanism underlying AD neuropathologies is debated.

Until recently, only a symptom-based assay as discussed in McKhann et al., above, was available diagnosing the presence of Alzheimer’ s disease in a living patient. More recently, beginning in April of 2012 with Eli Lilly’s Amyvid®, and later with approval of GE Healthcare’s Vizamyl® and Piramal Imaging’s Neuraceq ®, PET scanning technology has been used to assay for AD in a living human. The intravenous–infused, radiolabeled positronemitting compound binds to Aβ in brain plaques.

Although accurate, PET scanning assays are inconvenient for patients in that the patients have to place their heads in a relatively confined space within a scintillation detector and should remain relatively motionless. PET scanning assays are also costly, particularly as compared to a more usual blood test that one receives in which a few milliliters of blood is taken to provide for as many as 40 different assays that unfortunately do not yet commercially include a test for AD.

Discovered 45 years ago as the first non-muscle actin-binding protein [Hartwig et al., J Biol Chem 250:5696-5705 (1975); Wang et al., Proc Natl Acad Sci USA 72:4483-4486 (1975)], filamins [FLNs] are a family of cytoskeletal proteins - filamins A (FLNA) and B, but not C - that are expressed in non-muscle cells. Human FLNA is given the identifier P21333 in the UniProtKB/Swiss-Prot data base, and contains a sequence of 2647 amino acid residues (about 280 kDa). This protein is also sometimes referred to in the art as actin-binding protein (ABP-280). [Gorlin et al., J Cell Biol 111: 1089-1105 (1990).]

The FLNA protein anchors various transmembrane proteins to the actin cytoskeleton and serves as a scaffold for a wide range of cytoplasmic signalling proteins. Filamins are essential for mammalian cell locomotion and act as interfaces for protein-protein interaction [van der Flier et al., Biochim Biophys Acta 1538:99-117 (2001)]. Besides its role in cell motility, FLNA is increasingly found to regulate cell signalling by interacting with a variety of receptors and signalling molecules. [Stossel et al., Nat Rev Mol Cell Biol 2:138-145 (2001); Feng et al., Nat Cell Biol 6:1034-1038 (2004)).

The FLNA protein consists of an N-terminal actin-binding domain (ABD) and a rod-like domain of 24 immunoglobulin-like repeat domains (IgFLNa’s) (each about 96-amino acid residues long and numbered from the N-terminus), interrupted by two 30 \-amino acid residue flexible loops or hinges. The IgFLNa’s are numbered 1 through 24, beginning near the N-terminus and ending near the C-terminus. The loop designated H1 is between repeats 15 and 16, and the loop designated H2 is located between repeats 23 and 24 [Gorlin et al., J Cell Biol 111:1089-1105 (1990); van der Flier et al., Biochim Biophys Acta 1538:99-117 (2001)].

H1 and H2 can be cleaved by calpains and caspases [Gorlin et al., J Cell Biol 111:1089-1105 (1990); Browne et al., J Biol Chem 275:39262-39266 (2000)]. Cleavage at H1 occurs between ammo acid residues 1762 and 1764, and results in an about 170 kDa fragment consisting of the ABD and repeats 1-15 (IgFLNa-1-15), plus an about 110 kDa polypeptide fragment consisting of repeats 16-24 (IgFLNa-16-24).

It is noted that the UniProtKB/Swiss-Prot data base lists a C-terminus for repeat 15 at position 1740 and a N-terminus for repeat 16 as being located at amino acid residue position 1779. On the other hand, Gorlin et al., above, place the calpain cleavage site between residues 1762 and 1764, whereas Garcia et al., Arch Biochem Biophys 446: 140-150 (2006) places that site between residues 1761 and 1762. Similarly, Gorlin et al., above, write that earlier authors (Hartwig et al., J Cell Biol 87:841-848 (1980)] reported the full length FLNA molecule to have a molecular weight of 270 kDa at page 1089 and thereafter as having a 280 kDa protein at page 1093.

Garcia et al. above, reports that calpain cleaves full length FLNA into polypeptide fragments of 180, 100, 90 and 10 kDa, whereas Bedolla et al., Clin Cancer Res 15(3):788-796(2009) report proteolysis fragments of 170, 110 and a 90 kDa fragment cleaved from the 110 kDa fragment. Browne et al., above, reported that the cytotoxic T lymphocyte protease granzyme B, grB, cleaves filamin in concert with the lytic protein perforin, and that filamin is also cleaved in a caspase-dependent manner following ligation to a Fas receptor. Western blots of dying Jurkat cell lysates identified two caspase cleavage polypeptides from the C-terminal region of FLNA having masses of about 110 and 95 kDa were identified. Purified grB cleaved filamin into several polypeptides including those having masses of about 205,200 and 110 kDa. Polyclonal rabbit antibodies raised to a fusion protein containing 476 amino acid residues from the C-terminal region of FLNA (positions 2172-2647) were used. Umeda et al. J Biochem 130:535-542 (2001) found somewhat similar results (C-terminal 135, 120 and 110 kDa polypeptide fragments) in U937 monoblastic leukemia and Jurkat human T lymphoblastic cells for proteolysis by caspase-3.

It is noted that IgFLNa-16-24 is said to have a mass of about 110 kDa in Loy et al., Proc Natl Acad Sci, USA, 100(8):4562-4567 (2003). That about 110 kDa polypeptide (IgFLNa-16-24) is further cleaved at H2 by calpain with a longer digestion time to yield an about 90 kDa, fragment that contains repeats 16-23 (IgFLNa-16-23) [Gorlin et al., J Cell Biol 111 :-1089-1105 (1990); van der Flier et al., Biochim Biophys Acta 1538:99-117 (2001)].

Because of the differences in residue positions and some molecular weights for the full length FLNA and its proteolysis fragments reported in the art as discussed above, the full length FLNA molecule and the smaller FLNA cleavage product as having molecular weights of “about” 280 kDa and “about” 90 kDa, respectively.

FLNA promotes orthogonal branching of actin filaments and links actin filaments to membrane glycoproteins. Filamin A is dimerized through the carboxy-terminal repeat (repeat 24) near the transmembrane regions, providing an intracellular V-sliaped structure that is critical for function.

Each v-shaped FLNA dimer has two antiparallel self-bound domains 24 forming the apex of the “v”, and the remaining domains stretched out much like beads on a string with each of their N-terminal ABD portions bound to an actin molecule. More recently, it has been reported that C-terminus of the ABD, rod segment 1 (IgFLNa-1-15), forms an extended linear structure without obvious inter-domain interactions. Rod segment 2 (IgFLNa-16-23) assumes a compact structure due to multiple inter-domain interactions in which domains 16-17, 18-19 and 20-21 form paired structures. [Heikkinen et al., J Biol Chem, 284:25450-1-5458 (2009); Lad et al., EMBOJ, 26:3993--4004 (2007).]

Proteolysis of FLNA is regulated in part by its phosphorylation on Ser 2152 (S2152) in repeat 20 (IgFLNa-20), which is reported to render the full-length protein stable and resistant to cleavage. [Gorlin et al., J Cell Biol 111: 1089–1105 (1990); Garcia et al., Arch Biochem Biophys 446:140–150 (2006); and Chen et al., J Biol Chem 264(24):14282-14289 (1989).]

Loy et al., Proc Natl Acad Sci, USA, 100(8):4562-4567 (2003), report that a H1 cleavage product containing repeats 16-24 and having a molecular weight of about 100 kDa colocalized with the androgen receptor to the nucleus in prostate cancer cells. Those workers noted that FLNA is generally regarded as a cytoplasmic architectural molecule, and characterized their finding of an additional function of its about 100 kDa polypeptide as is produced by calpain cleavage as a nuclear regulator of the androgen receptor to be “entirely unexpected” (at page 4565).

The about 100 kDa FLNA fragment found in a cellular nucleus is not phosphorylated on S2152. Indeed, phosphorylation on S2152 is reported to block the cleavage of full length FLNA by calpain in a prostate cancer line and in platelets. [Garcia et al., Arch Biochem Biophys 446:140-150 (2006); and Chen et al., J Biol Chem 264(24):14282-14289 (1989)].

Wang et al., Oncogene 26:6061-6070 (2007), showed that nuclear localization of FLNA correlates with hormone-dependence in prostate cancer. The non-phosphorylated about 90 kDa fragment (IgFLNa-16-23) migrates to the nucleus of hormone-naive cells in the androgen-dependent form of the disease. Contrarily, in hormone-refractory androgenindependent prostate tumor cells, FLNA was phosphorylated, preventing its cleavage and nuclear translocation. Those authors and co-workers subsequently showed that not only does prostate cancer metastasis correlate with cytoplasmic localization of FLNA, but that metastasis can be prevented by cleavage and subsequent nuclear translocation of the phosphorylated protein. [Bedolla et al., Clin Cancer Res. 15(3):788–796 (2009).]

As a key regulator of the cytoskeleton network, FLNA interacts with many proteins involved in cancer metastasis, [Yue et al., Cell & Biosci 3:7 (2013)] as well as in many other conditions. Thus, Nakamura et al., Cell Adh Migr. 5(2):160–169 (2011), discuss the history of research concerning FLNA and note that the protein serves as a scaffold for over 90 binding partners including channels, receptors, intracellular signalling molecules and transcription factors.

FLNA also has been implicated in tumor progression. FLNA knockout mice show reduced oncogenic properties of K-Ras, including the downstream activation of ERK and Akt. [Nallapalli et al., Mol Cancer 11:50 (2012).] Many different cancers show high levels of FLNA expression in contrast to low FLNA levels in corresponding normal tissue, including colorectal and pancreatic cancers, [Uhlen et al., Mol Cell Proteomics 4:1920-1932 (2005)] and glioblastoma [Sun et al., Cancer Cell 9:287-300 (2006)).

Inhibition of FLNA expression sensitizes cancer cells to both cisplatin and radiation [Sun et al., Cancer Cell 9:287-300 (2006)], and FLNA deficiency in cancer cells similarly sensitizes them to chemotherapeutic agents [Yue et al., DNA Repair (Amst) 11:192-200 (2012)] and radiation [Yue et al., Cancer Res 69:7978-7985 (2009); Yuan et al., J Biol Chem 276:48318-48324 (2001)]. On the other hand, Jiang et al., Int. J. Biol. Sci. 9:67-77 (2013) report that inhibition of filamin A expression leads to reduced metastasis in nude mice implanted with melanoma and breast cancer cells.

Phosphorylation has become recognized as a global regulator of cellular activity, and abnormal phosphorylation is implicated in a host of human diseases, particularly cancers. Phosphorylation of a protein involves the enzymatically-mediated replacement of an amino acid side chain hydroxyl of one or more serine, threonine or tyrosine residues with a phosphate group (-OPO₃⁻²).

Phosphorylation and its reverse reaction, dephosphorylation, occur via the actions of two key enzyme types. Protein kinases phosphorylate proteins by transferring a phosphate group from a nucleotide triphosphate such as adenosine triphosphate (ATP) or guanosine triphosphate (GTP) to their target protein. This process is balanced by the action of protein phosphatases, which can subsequently remove the phosphate group.

The amount of phosphate that is bonded to a protein at a particular time is therefore determined by the relative activities of the particular one or more associated kinase and phosphatase enzymes specific to that protein and to the particular amino acid residue(s) undergoing phosphorylation/ dephosphorylation. If the phosphorylated protein is an enzyme, phosphorylation and dephosphorylation can impact its enzymatic activity, essentially acting like a switch, turning it on and off in a regulated manner. Phosphorylation can similarly regulate non-enzymatic protein-protein interactions by facilitation of binding to a partner protein.

Protein phosphorylation can have a vital role in intracellular signal transduction. Many of the proteins that make up a signaling pathway are kinases, from the tyrosine kinase receptors at the cell surface to downstream effector proteins, many of which are serine/threonine kinases.

FLNA is phosphorylated at a number of positions in its protein sequence in both normal and in diseased cells such as cancer cells. For example, the enzyme PAK1 (EC 2.7.11.1) is a protein kinase of the STE20 family that regulates cell motility and morphology. FLNA phosphorylation at position 2152 by PAK1 is required for PAK1-mediated actin cytoskeleton reorganization and for PAK1-mediated membrane ruffling. [Vadlamudi et al., Nat. Cell Biol. 4:681-690 (2002); Woo et al., Mol Cell Biol. 24(7):3025–3035 (2004).] Cyclin Bl/Cdkl (EC:2.7.11.22; EC:2.7.11.23) phosphorylates serine 1436 in vitro in FLNA-dependent actin remodeling. [Cukier et al., FEBS Letters 581(8):1661-1672 (2007).]

The UniProtKB/Swiss-Prot data base entry for human FLNA (No. P21333) lists published reports of the following amino acid residue positions as being phosphorylated under different circumstances: 11, 1081, 1084, 1089, 1286, 1338, 1459, 1533, 1630, 1734, 2053, 2152, 2158, 2284, 2327, 2336, 2414, and 2510. Further, polyclonal and monoclonal antibodies are commercially available from one or more of Abgent, Inc. (San Diego, CA), Abcam® Inc. (Beverly, MA), Bioss, Inc. (Woburn, MA), and GeneTex, Inc. (Irvine, CA) that immunoreact with FLNA that is phosphorylated (phospho-FLNA) at serine-1083, tyrosine-1046, serine-1458, serine-2152, and serine-2522.

The 90 kDa FLNA fragment that can localize to the nucleus and interact with transcription factors includes the serine-2152 residue that can be phosphorylated. However, that previously discussed nucleus-localized about 90 kDa FLNA fragment is free of phosphorylation at serine-2152. Indeed, phosphorylation of FLNA at serine-2152 (“pS2152” FLNA) has been reported to protect FLNA from proteolysis to form the about 90 kDa fragment [Garcia et al., Arch Biochem Biophys 446: 140-150 (2006); Gorlin et al., J Cell Biol 111:1089-1105 (1990); and Chen et al., J Biol Chem 264(24):14282-14289 (1989)].

It has been shown that the toxic signalling of amyloid-β42 (Aβ42) by the α7~ nicotinic acetylcholine receptor (α7nAChR), which results in tau phosphorylation and formation of neurofibrillary tangles associated with Alzheimer’s disease, requires the recruitment of the scaffolding protein FLNA to activate TLR4 through CD14, Wang et al., J. Neurosci. 32(29):9773-9784 (Jul. 18, 2012). That paper also showed that PTl~125 (simufilam) provides an anti-inflammatory effect by similarly reducing FLNA association with toll-like receptor 4 (TLR4) and preventing cytokine release.”

### BRIEF SUMMARY OF THE INVENTION

Scaffolding protein FLNA is recognized as having an association with the development of Alzheimer’s disease through its involvement of Aβ42 signalling through α7nAChR, and the abnormally folded form of FLNA believed to be the linking agent in this process is the target of PTI-125 (simufilam), a small molecule antagonist of FLNA currently being investigated for use in treatment of AD. Interestingly, FLNA is also strongly expressed in platelets where it is thought to regulate normal platelet functions, and mutations in FLNA can cause platelet-related disorders. Platelets also express functional α7nAChR (Schedel et al., ArteriosclThrom, Vas, 31:928-934 (2011.)). There have also been reports that platelets can shuttle Aβ42 to the brain (Catricala et al., ImmunAgeing9:20 (2012). FLNA is also known to be processed in platelets during activation [Buitrago et al.,bioRxiv 307397] and this can assist in creation of abnormally folded forms of FLNA able to bind Aβ42 and/or α7nAChR both within platelets and in the brain following platelet lysis.

Current research has failed to produce viable diagnostic assays based on FLNA. Indeed, current diagnostic methods for Alzheimer’s disease typically require tissue samples (e.g., for post-mortem confirmation) or cerebrospinal fluid obtained via invasive surgical procedures. The present disclosure addresses these and other shortcomings associated with current assays by providing new blood-based assays and methods that can be used to diagnose AD and other tauopathies, as well as kits and reagents related to the same based on novel biomarkers (e.g., FLNA having a particular phosphorylation profile) or fragments thereof, for use in methods for diagnosing, staging, treating and assessing the response of a treatment for neurocognitive disorders such as Alzheimer’s disease. In addition, the present invention provides novel targets for the development of new therapies against tauopathies or for repurposing existing therapies not originally designed for the treatment of neurocognitive disorders such as tauopathies.

In a first general aspect, the present disclosure provides a biomarker panel comprising one or more phosphorylated peptides obtained from in vitro digestion of Filamin A (SEQ ID NO: 1). In some aspects, the FLNA is contained within or obtained from a biological fluid or tissue sample taken from a subject suspected of having a neurological disease (e.g., Alzheimer’s disease or another tauopathy). For the sake of brevity, example sequences are given based on digestion with the proteases trypsin, GluC and AspN. The skilled practitioner would understand that digestion with other proteases generate alternative peptide sequences comprising the same phosphorylated residues and all such alternatives are encompassed within the present specification.

In some aspects, the phosphorylated peptide is phosphorylated at residue serine 2152 of SEQ ID NO: 1. In some aspects, the phosphorylated peptide has the amino acid sequence of SEQ ID NO: 2, SEQ ID NO: 3, SEQ ID NO: 4, or SEQ ID NO: 5 (i.e., a fragment of full-length FLNA). In some aspects, the biomarker panel can comprise a plurality of phosphorylated fragments of FLNA, e.g., two or more phosphorylated peptides, each comprising a fragment of FLNA and having the amino acid sequence of any one of SEQ ID NOs: 2-5, wherein at least one of the two or more phosphorylated peptides is phosphorylated at a position corresponding to serine 2152 of SEQ ID NO: 1 (full-length FLNA).

In some aspects, the phosphorylated peptide is phosphorylated at residue serine 2143 of SEQ ID NO: 1. In some aspects, the phosphorylated peptide has the amino acid sequence of SEQ ID NO: 6, SEQ ID NO: 7, SEQ ID NO: 8, or SEQ ID NO:9 (i.e., a fragment of full-length FLNA). In some aspects, the biomarker panel can comprise a plurality of phosphorylated fragments of FLNA, e.g., two or more phosphorylated peptides, each comprising a fragment of FLNA and having the amino acid sequence of SEQ ID NOs: 6-9, wherein at least one of the two or more phosphorylated peptides is phosphorylated at a position corresponding to serine 2143 of SEQ ID NO: 1 (full-length FLNA).

In some aspects, the phosphorylated peptide is phosphorylated at residue serine 2180 of SEQ ID NO: 1. In some aspects, the phosphorylated peptide has the amino acid sequence of SEQ ID NO: 10, SEQ ID NO: 11, or SEQ ID NO: 12 (i.e., a fragment of full-length FLNA). In some aspects, the biomarker panel cancomprise a plurality of phosphorylated fragments of FLNA, e.g., two or more phosphorylated peptides, each comprising a fragment of FLNA and having the amino acid sequence of SEQ ID NOs: 10-12, wherein the two or more phosphorylated peptides are phosphorylated at a position corresponding to residue serine 2180 of SEQ ID NO: 1 (full-length FLNA).

In some aspects, the phosphorylated peptide is phosphorylated at residue serine 1459 of SEQ ID NO: 1. In some aspects, the phosphorylated peptide has the amino acid sequence of SEQ ID NO: 13, SEQ ID NO: 14, or SEQ ID NO: 15 (i.e., a fragment of full-length FLNA). In some aspects, the biomarker panel can comprise a plurality of phosphorylated fragments of FLNA, e.g., two or more phosphorylated peptides, each comprising a fragment of FLNA and having the amino acid sequence of SEQ ID NOs: 13-15, wherein the two or more phosphorylated peptides are phosphorylated at a position corresponding to residue serine 1459 of SEQ ID NO: 1 (full-length FLNA).

In some aspects, the biomarker panel comprises a plurality of phosphorylated peptides, wherein each phosphorylated peptide is a fragment of FLNA (SEQ ID NO: 1) and comprises a phosphorylation site at a position corresponding to serine 1459, 2143, 2152, and/or 2180 of full-length FLNA (SEQ ID NO:1). For example, the biomarker panel can comprise a plurality of phosphorylated peptides, each having the sequence of SEQ ID Nos: 2-15 and being phosphorylated at a position corresponding to serine 1459, 2143, 2152, and/or 2180 of full-length FLNA (SEQ ID NO: 1). In some aspects, the biomarker panel can comprise one or more phosphorylated peptides which comprise a fragment of full-length FLNA that is phosphorylated at two or more of the above-identified phosphorylation sites (e.g., at serine 1459, 2143, 2152, and/or 2180 of full-length FLNA).

In some aspects, the biomarker panel comprises at least 2, 3, 4, 5, 6, 7, 8, 9, or 10 different phosphorylated peptides, each being phosphorylated at a site corresponding to serine residue 1459, 2143, 2152 and/or 2180 of full-length FLNA (SEQ ID NO: 1).

In some aspects, the biomarker panel further comprises one or more peptides listed in Table 4, which can be obtained from in vitro digestion of Filamin A using a protease or combination of proteases, wherein each of the one or more peptides comprises an amino acid sequence present in SEQ ID NO:1. In some aspects, the peptide is proteotypic for FLNA.

In a second general aspect, the disclosure provides a panel of biomarkers, comprising: (i) one or more peptides obtained from in vitro digestion of Filamin A using a protease or combination of proteases, wherein each of the one or more peptides comprises an amino acid sequence present in SEQ ID NO:1; and (ii) one or more peptides obtained from in vitro digestion of one or more, optionally two or more of the proteins listed in Table 1. In some aspects, the one or more peptides are obtained from in vitro digestion with one or a combination of proteases selected from trypsin, GluC, ArgC, AspN, and/or chymotrypsin. In some aspects, the Filamin A and one or more proteins listed in Table 1 are contained within a biological fluid or tissue sample obtained from a subject suspected of having a neurological disease. In some aspects, the neurological disease is Alzheimer’s disease or another tauopathy. In some aspects, the one or more peptides obtained from in vitro digestion of Filamin A comprise one or more phosphorylation sites. In some aspects, the one or more phosphorylation sites correspond to serine residue 2143, 2152 and/or 2180 of full-length FLNA (SEQ ID NO: 1).

In a third general aspect, the disclosure provides a panel of biomarkers, comprising: (i) one or more peptides obtained from in vitro digestion of Filamin A using a protease or combination of proteases, wherein each of the one or more peptides comprises an amino acid sequence present in SEQ ID NO: 1; and (ii) one or more peptides obtained from in vitro digestion of one or more, optionally two or more, of the proteins listed in Table 2. In some aspects, the one or more peptides are obtained from in vitro digestion with one or a combination of proteases selected from trypsin, GluC, ArgC, AspN, and/or chymotrypsin. In some aspects, the Filamin A and one or more proteins listed in Table 2 are contained within a biological fluid or tissue sample obtained from a subject suspected of having a neurological disease. In some aspects, the neurological disease is Alzheimer’s disease or another tauopathy. In some aspects, the one or more peptides obtained from in vitro digestion of Filamin A comprise one or more phosphorylation sites. In some aspects, the one or more phosphorylation sites correspond to serine residue 2143, 2152 and/or 2180 of full-length FLNA (SEQ ID NO: 1).

In a fourth general aspect, the disclosure provides a panel of biomarkers, comprising: (i) one or more peptides obtained from in vitro digestion of Filamin A using a protease or combination of proteases, wherein each of the one or more peptides comprises an amino acid sequence present in SEQ ID NO: 1; and (ii) one or more peptides obtained from in vitro digestion of one or more, optionally two or more, of the proteins listed in Table 3. In some aspects, the one or more peptides are obtained from in vitro digestion with one or a combination of proteases selected from trypsin, GluC, ArgC, AspN, and/or chymotrypsin. In some aspects, the Filamin A and one or more proteins listed in Table 3 are contained within a biological fluid or tissue sample obtained from a subject suspected of having a neurological disease. In some aspects, the neurological disease is Alzheimer’s disease or another tauopathy. In some aspects, the one or more peptides obtained from in vitro digestion of Filamin A comprise one or more phosphorylation sites. In some aspects, the one or more phosphorylation sites correspond to serine residue 2143, 2152 and/or 2180 of full-length FLNA (SEQ ID NO: 1).

In a fifth general aspect, the disclosure provides a panel of biomarkers, comprising: (i) one or more peptides obtained from in vitro digestion of Filamin A using a protease or combination of proteases, wherein each of the one or more peptides comprises an amino acid sequence present in SEQ ID NO: 1; and (ii) one or more peptides obtained from in vitro digestion of one or more, optionally two or more of the proteins listed in Tables 1, 2, 3, or 4. In some aspects, the one or more peptides are obtained from in vitro digestion with one or a combination of proteases selected from trypsin, GluC, ArgC, AspN, and/or chymotrypsin. In some aspects, the Filamin A and one or more proteins listed in Table 1 are contained within a biological fluid or tissue sample obtained from a subject suspected of having a neurological disease. In some aspects, the neurological disease is Alzheimer’s disease or another tauopathy.

In a sixth general aspect, the disclosure provides a panel of biomarkers, comprising: one or more synthetic peptides, wherein one or more of the synthetic peptides are enriched with heavy isotopes of H, C, N, O and/or S. In some aspects, one or more of the synthetic peptides comprises an amino acid sequence present in in Filamin A (SEQ ID NO: 1); or present in one of the proteins listed in Tables 1, 2, 3, or 4. In some aspects, one or more of the synthetic peptides comprises an amino acid sequence corresponding to the sequence of a peptide obtained from in vitro digestion of SEQ ID NO: 1 or of one of the proteins listed in Tables 1, 2, 3, or 4, using one or a combination of proteases selected from trypsin, GluC, ArgC, AspN, and/or chymotrypsin.

In a seventh general aspect, the disclosure provides a method for the diagnosis/prognosis of a neurological disorder, comprising: obtaining a bodily fluid or tissue sample from a subject (optionally, a subject suspected of having a neurological disorder such as Alzhiemer’s disease); digesting one or more proteinaceous materials (e.g., proteins and/or polypeptides) in the bodily fluid or tissue sample with one or more proteases (e.g., trypsin, GluC, ArgC, AspN, and/or chymotrypsin); and detecting and/or measuring the level of one or more peptides produced from the digestion, using mass spectrometry. In some aspects, each of the one or more peptides comprises an amino acid sequence corresponding to the sequence of a peptide obtained from in vitro digestion of Filamin A (SEQ ID NO: 1) or of one of the proteins listed in Tables 1, 2, or 3.

In an eighth general aspect, the disclosure provides a method for monitoring the progression of a neurological disorder, comprising: (a) obtaining a bodily fluid or tissue sample from a subject (optionally, a subject suspected of having a neurological disorder such as Alzhiemer’s disease) at a first time point; (b) digesting one or more proteins in the bodily fluid or tissue sample with one or more proteases (e.g., trypsin, GluC, ArgC, AspN, and/or chymotrypsin); (c) detecting and/or measuring the level of one or more peptides produced from the digestion, using mass spectrometry; and (d) repeating steps (a)-(c) at a second time point. In some aspects, the method further comprises step (e) of determining whether a therapeutic treatment administered to the subject is effective in treating the neurological disorder based upon the detected and/or measured levels of the one or more peptides of step (c). In some aspects, each of the one or more peptides comprises an amino acid sequence corresponding to the sequence of a peptide obtained from in vitro digestion of Filamin A (SEQ ID NO: 1) or of one of the proteins listed in Tables 1, 2, or 3.

In a ninth general aspect, the disclosure provides a method for diagnosing or monitoring the progression of a neurological disorder (such as Alzheimer’s disease or another tauopathy), comprising: obtaining a bodily fluid or tissue sample from the subject; digesting one or more proteins in the bodily fluid or tissue sample with one or more proteases (e.g., trypsin, GluC, ArgC, AspN, and/or chymotrypsin); and measuring the level if any of one or more phosphorylated peptide fragments produced from the digestion of Filamin A (SEQ ID NO:1). In some aspects, the one or more fragments comprise a phosphorylated serine at a position corresponding to serine 2152 and/or 2143 of Filamin A (SEQ ID NO:1).

In some aspects, the method further comprises determining a ratio of two or more of the above peptide fragments. In some aspects, the ratio comprises a ratio of fragments that are phosphorylated at a position corresponding to serine 2143 of Filamin A (SEQ ID NO:1) versus fragments that are phosphorylated at a position corresponding to serine 2152 of Filamin A (SEQ ID NO:1). In some aspects, the ratio is determined using two or more peptides that each comprise a sequence of SEQ ID Nos: 2-9. In some aspects, the ratio comprises a ratio of fragments that are phosphorylated at a position corresponding to serine 2143 of Filamin A (SEQ ID NO: 1) versus fragments which are phosphorylated at a position corresponding to serine 2152 of Filamin A (SEQ ID NO:1); wherein a ratio of <5 indicates that the subject does not have Alzheimer’s disease and a ratio of >10 indicates that the subject has Alzheirner’s disease.

In a tenth general aspect, the disclosure provides a method for diagnosing or monitoring the progression of a neurological disorder (such as Alzheimer’s disease or another tauopathy), comprising: obtaining a bodily fluid or tissue sample from the subject; digesting one or more proteins in the bodily fluid or tissue sample with one or more proteases (e.g., trypsin, GluC, ArgC, AspN, and/or chymotrypsin); and measuring the level of any of one or more phosphorylated peptides produced from the digestion of Filamin A (SEQ ID NO: 1) and one or more peptides derived from Integrin alpha-IIb, Integrin beta-3, and/or a Linker for activation of T-cells family member 1, wherein the presence of Integrin alpha-IIb, Integrin beta-3, and/or Linker for activation of T-cells family member 1, indicates ex vivo or artefactual platelet activation.

### BRIEF DESCRIPTION OF THE DRAWINGS

FIG. 1 shows a representation of the TMTcalibrator™ mass spectrometry analysis workflow. After depletion of the top 14 abundant proteins, plasma samples were digested and labelled with TMTpro™ reagents. In parallel, brain lysates were also digested and labelled with TMTpro™ reagents. Plasma and brain lysate digests were then mixed and a small aliquot analysed for total proten expression by tandem mass spectrometry (LC-MS/MS). The phosphopeptide fraction was enriched from the remaining mixture and analysed by LC-MS/MS. Data analysis identified peptide sequences and relative abundances based on TMTpro™ reporter ions and linear modelling used to identify features showing differential expression in AD and Control samples.

FIG. 2 shows a histogram of the expression profile for FLNA in 6 AD plasma samples prepared on HistopaqueⓇ-1077 and 6 plasma samples collected in EDTA tubes. Values are expressed as log2 ratios relative to the reference brain lysate channel.

FIG. 3 shows a histogram of the expression profile for the tryptic peptide APsVANVGSHCDLSLK phosphorylated at serine 2152 of FLN.A (SEQ ID NO: 1) in 6 AD plasma samples prepared on HistopaqueⓇ-1077 and 6 plasma samples collected in EDTA tubes. Values are expressed as isotope-corrected TMTpro™ reporter ion intensities.

FIG. 4 shows a histogram of the expression profile for the tryptic peptide RAPsVANVGSJCDLSLK phosphorylated at serine 2152 of FLNA (SEQ ID NO: 1) in 6 AD plasma samples prepared on Histopaque®-1077 and 6 plasma samples collected in EDTA tubes. Values are expressed as isotope-corrected TMTpro™ reporter ion intensities.

FIG. 5 shows a histogram of the expression profile for the tryptic peptide VKEsITR phosphorylated at serine 2143 of FLNA (SEQ ID NO: 1) in 6 AD plasma samples prepared on HistopaqueⓇ-1077 and 6 plasma samples collected in EDTA tubes. Values are expressed as isotope-corrected TMTpro™ reporter ion intensities.

FIG. 6 shows a histogram of the expression profile for the tryptic peptide IPEISIQDMTAQVTsPSGK phosphorylated at serine 2180 of FLNA (SEQ ID NO: 1) in 6 AD plasma samples prepared on HistopaqueⓇ-1077 and 6 plasma samples collected in EDTA tubes. Values are expressed as isotope-corrected TMT’pro™ reporter ion intensities.

FIGS. 7A, 7B, 7C, and 7D) show four box-and-whisker plots representing the relative expression of the four FLNA phosphopeptides RAPsVANVGSHCDLSLK(SEQ ID NO: 3; FIG. 7A), IPEISIQDMTAQVTsPSGK (SEQ ID NO: 10; FIG. 7B), VKEsITR (SEQ ID NO: 6; FIG. 7C) and CSGPGLsPGMVR (SEQ ID NO: 13; FIG. 7D) in 6 AD plasma samples prepared on Histopaque®-1077 and 6 plasma samples collected in EDTA tubes. The values shown are log2 ratios relative to the reference brain lysate channel.

FIGS. 8A, 8B, and 8C show heatmaps of log2 transformed phosphopeptide expression levels relative to the brain lysate channel in 6 AD plasma samples prepared on HistopaqueⓇ-1077 and 6 plasma samples collected in EDTA tubes. The parent gene names of the regulated phosphopeptides are shown to the right of the figure in each panel. FIG. 8A shows regulated phosphopeptides in AD cases versus old controls. FIG. 8B shows regulated phosphopeptides in AD cases versus young controls. FIG. 8C shows regulated phosphopeptides in AD cases versus all controls.

FIGS. 9A, 9B, and 9C provide histograms showing the expression profile for the tryptic peptides VKE[p]sITRphosphorylated at serine 2143 of FLNA (SEQ ID NO: 1) (FIG. 9A), RAP[p]sVANVGSHCDLSLK phosphorylated at serine 2152 of FLNA (SEQ ID NO: 1) (FIG. 9B), and AP[p]sVANVGSHCDLSLK (SEQ ID NO: 138) phosphorylated at serine 2152 of FLNA (SEQ ID NO: 1) (FIG. 9C); in 6 AD plasma samples prepared on Histopaque®-1077 and 6 plasma samples collected in EDTA tubes. Values are expressed as isotope-corrected TMTpro™ reporter ion intensities.

FIGS. 10A, 10B, 10C, and 10D show four box-and-whisker plots representing the relative expression of the four FLNA phosphopeptides, AP[p]sVANVGSHCDLSLK (SEQ ID NO: 1; FIG. 10A) RAP[p]sVANVGSHCDLSLK (SEQ ID NO: 3, FIG. 10B), rL[p]tVSSLQESGLk (SEQ ID NO: 186; FIG. 10C), and CSGPGL[p]sPGMVR (SEQ ID NO: 13; FIG. 10D) in 6 AD plasma samples prepared on HistopaqueⓇ-1077 and 6 plasma samples collected in EDTA tubes. The values shown are log2 ratios relative to the reference brain lysate channel.

### DETAILED DESCRIPTION OF THE INVENTION

For purposes of interpreting this specification, the following definitions and abbreviations will apply unless specified otherwise and whenever appropriate, terms used in the singular will also include the plural and vice versa.

The term “biomarker(s)” includes all biologically relevant forms of the protein identified, including post-translational modifications. For example, the biomarker can be present in a glycosylated, phosphorylated, multimeric, fragmented or precursor form. A biomarker fragment can be naturally occurring or, for example, enzymatically generated and still retaining the biologically active function of the full protein. Fragments will typically be at least about 10 amino acids, usually at least about 50 amino acids in length, and can be as long as 300 amino acids in length or longer.

The term “canonical sequence” is used herein as to refer to the most prevalent sequence and/or the most similar sequence among orthologous species. In particular, unless otherwise specified, the canonical sequence refers herein to the human sequence.

The peptide sequences disclosed herein are represented using the IUPAC single-letter code. Use of lower-case letters indicate a modification as follows: “c” —carbamidomethylated cystine; “m” - oxidized methionine; “n” — de-amidated asparagine; “q” - de-amidated glutamine; and “k” - TMT-labelled lysine. A lower case letter at the N-terminus represents a TMT-modified amino acid. In addition, the symbol “[p]” is used to indicate phosphorylation on the succeeding amino acid, e.g., “[p]s” denotes a phosphorylated serine; “[p]t” denotes a phosphorylated threonine; and “[p]y” denotes a phosphorylated tyrosine.

The term “KEGG pathway” refers to a collection of manually drawn pathway maps representing molecular interactions and reaction networks for metabolism, genetic information processing, environmental information processing, cellular processes, organismal systems, human diseases and drug development. “KEGG pathways mapping” is the process to map molecular datasets, especially large-scale datasets in genomics, transcriptomics, proteomics, and metabolomics, to the KEGG pathway maps for biological interpretation of higher-level systemic functions; (http://www.genome.jp/kegg/pathway.html).

The term “concentration or amount” refers to the relative concentration or amount of biomarker in the sample, for example as determined by LC-MS/MS label free quantification approaches such as area under the curve and spectral counting.

The term “comparing” or “compare” or grammatical equivalents thereof, means measuring the relative concentration or amount of a biomarker in a sample relative to other samples (for example protein concentrations or amounts stored in proprietary or public database).

The term “reference concentration or amount” refers to, but it is not limited to, protein concentrations or amounts stored in proprietary or public databases. The “reference concentration or amount” can have been obtained from a large screening of patients, or by reference to a known or previously determined correlation between such a determination and clinical information in control patients. For example, the reference values can be determined by comparison to the concentration or amount of the biomarkers in a control subject, for example a healthy person (i.e. without dementia) of similar age and gender as the subject. Alternatively, the reference values are values that can be found in literature such as the A.poE 24 allele presence whereby the presence or absence of the mutations at position 112 and 158 represent the reference to be compared to, or like the levels of total tau (T-tau) >350 ng/L, phospho-tau (P-tau) >80 ng/L and A~42 <530 ng/L in the CSF (Hansson et al., Lancet Neural. 5(3):228-234 (2006). In addition, the reference values can have been obtained from the same subject at one or more time points that precede in time the test time point. Such earlier samples can be taken one week or more, one month or more, three months or more, most preferably six months or more before the date of the test time point. In some embodiments, multiple earlier samples can be compared in a longitudinal manner and the slope of change in biomarker expression, if any, can be calculated as a correlate of cognitive change, such as the usually noted decline.

The term “control” or as used herein “non AD control” or “non AD subject” refers to a tissue sample or a bodily fluid sample taken from a human or non-human subject that is cognitively normal or diagnosed with or presenting symptoms of a cognitive abnormality but defined, with respect to the existing biochemical tests, as non AD subjects.

The terms “selected reaction monitoring”, “SRM” and “MRM” mean a mass spectrometry assay whereby precursor ions of known mass-to-charge ratio representing known biomarkers are preferentially targeted for analysis by tandem mass spectrometry in an ion trap or triple quadrupole mass spectrometer. During the mass spectral analysis, the parent ion is fragmented and the number of selected daughter ions of a second predefined mass-to-charge ratio is counted. Typically, an equivalent precursor ion bearing a predefined number of stable isotope substitutions but otherwise chemically identical to the target ion is included in the method to act as a quantitative internal standard.

The terms “parallel reaction monitoring” and “PRM” mean a mass spectrometry assay whereby precursor ions of known mass-to-charge ratio representing known biomarkers are preferentially targeted for analysis by tandem mass spectrometry in an Orbitrap™ mass spectrometer (Thermo Fisher Scientific, Waltham, MA). During the analysis, the parent ion is fragmented and the number of each of the daughter ions is counted. Typically, an equivalent precursor ion bearing a predefined number of stable isotope substitutions but otherwise chemically identical to the target ion is included in the method to act as a quantitative internal standard.

The term “proteotypic” means a peptide that is uniquely representative of the protein from which it is derived such that the sequence of amino acid residues in the peptide is not found in any other protein from the same species, apart from other expressed isoforms or splice variants derived from the same gene.

The term “phosphopeptide” means a peptide that contains at least one amino acid modified by addition of a phosphate group.

The term “isolated”, or grammatical equivalents thereof, means throughout this specification, that the protein, peptide, antibody, polynucleotide or chemical molecule as the case can be, exists in a physical milieu distinct from that in which it can occur in nature.

As used herein, the term “subject” includes any human or non-human animal.

The term “non-human animal” includes all vertebrates, e.g., mammals and non-mammals, such as non-human primates, rodents, sheep, dogs, cats, horses, cows, chickens, amphibians, reptiles, etc.

The term “treat”, “treating”, “treatment”, “prevent”, “preventing” or “prevention”, or grammatical equivalents thereof, includes therapeutic treatments, prophylactic treatments and applications in which one reduces the risk that a subject will develop a disorder or other risk factor. Treatment does not require the complete curing of a disorder and encompasses the reduction of the symptoms or underlying risk factors.

The term “diagnosis”, or grammatical equivalents thereof, as used herein, includes the provision of any information concerning the existence or non-existence or absence or probability of the disorder in a patient. It further includes the provision of information concerning the type or classification of the disorder or of symptoms that are or can be experienced in connection with it. This can include, for example, diagnosis of the severity of the disorder. The term “diagnosis” encompasses prognosis of the medical course of the disorder, for example its duration, severity and the course of progression from mild cognitive impairment (MCI) to AD or other dementias.

The term “staging”, or grammatical equivalents thereof, as used herein, means identifying in a subject the stage of a neurocognitive disorder, in particular AD. For example, AD is characterised by 3 stage or 7 stages, depending on the diagnostic framework used. The Global Dementia Scale is one such measure of global function. It is measured by assessment of severity including cognition and function against a standardised set of severity criteria.

The term “efficacy” indicates the capacity for beneficial change of a given intervention (e.g. a drug, medical device, surgical procedure, etc.) If efficacy is established, that intervention is likely to be at least as good as other available interventions, to which it has been compared. The term “efficacy” and “effectiveness” are used herein interchangeably.

The term “comprising” indicates that the subject includes all the elements listed, but can, optionally, also include additional, unnamed elements.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein,

Unless the context dictates otherwise, the definitions of the features/terms set out above are not limited to any particular aspect or embodiment of the invention and apply equally to all aspects and embodiments which are described herein.

The following abbreviations shall be understood as following in the context of the specification: CSF (cerebrospinal fluid); LBD (Lewy body dementia); FTD (fronto-temporal dementia); VaD (vascular dementia); ALS (amyotrophic lateral sclerosis); CJD (Creutzfeldt-Jakob disease); CNS (central nervous system); TMT® (Tandem Mass Tag®); TEAB (Tetra-ethylamonium Bicarbonate); TFA (Trifluoroacetic acid); SDS (Sodium dodecyl sulfate); TCEP (Tris(2-carboxyethyl)phosphine); ACN (Acetonitrile); Da (Dalton); HPLC (High-performance liquid chromatography); FA (Formic acid); LC-MS/MS (Liquid Chromatography with tandem Mass Spectrometry detection); MS (Mass Spectrometry); MS/MS or MS2 (Tandem MS); MS/MS/MS or MS3 (triple MS); PAGE (Polyacrylamide gel electrophoresis); SCX (Strong Cation Exchange); ppm (Parts per million); TiO2 (titanium dioxide); IMAC (iron metal affinity chromatography).

In AD pathogenesis, accumulation of the amyloid-6 peptide (AB) interacts with the signalling pathways that regulate the phosphorylation of tau. Hyperphosphorylation of tau disrupts its normal function in regulating axonal transport and leads to the accumulation of neurofibrillary tangles and toxic species of soluble tau. Currently there is no cure for AD.

Approved treatments are few and of limited efficacy for AD, serving mostly to slow or delay progression. Identification and development of new therapies for the treatment of AD and other tauopathies are also greatly affected by the lack of effective diagnostic, prognostic and predictive biomarkers and by the lack of new targets for the design of new therapies. Currently, AD can only be definitively diagnosed by brain biopsy or upon autopsy after a patient has died. Clearly, in clinical settings, brain biopsy is rarely performed and diagnosis is still primarily made based on the history of the symptoms and depends on a battery of neurological, psychometric and biochemical tests.

These latter tests include assessment of ApoE e4 allele status and measurements of amyloid beta, tau and phospho-tau in cerebrospinal fluid. These present methods, nevertheless, are still unsatisfactory not only for the early diagnosis of AD and other tauopathies, but also for predicting the progression of neurological disease that is important for recruitment of patients for clinical trials, for designing new therapies and for predicting the effectiveness of current and new therapies. Accordingly, the present disclosure provides new blood-based assays and methods that can be used to diagnose AD and other tauopathies and to monitor or predict the progression of these diseases (e.g., to guide treatment decisions).

The ideal diagnostic biomarkers should have high specificity for disease versus non-disease and high sensitivity to distinguish between disease types and stages. Prognostic biomarkers should reflect the intensity and severity of the pathological changes and predict their future course from a very early stage of the disease, before degeneration is observed, until advanced stages of the disease. Pharmacodynamic biomarkers should give a reliable indication whether an administered therapy is efficacious based on the changes in the level of disease-related proteins in readily accessible body fluids such as blood, blood products including platelets, serum and most preferably plasma and CSF. It is also desirable that such pharmacodynamics biomarkers can provide guidance to clinicians when to stop treatment or switch to a different therapy.

New targets need to be efficacious, safe, meet clinical and commercial needs and, above all, be “druggable.” A “druggable” target is accessible to the putative drug molecule, be that a small molecule or larger biologicals and upon binding, elicit a biological response that can be measured both in vitro and in vivo. In other words, its inhibition or activation results in a therapeutic effect in a disease state. Hence, there remains a need for proteins and/or peptides that can perform with superior sensitivity and/or specificity as biomarkers in the diagnosis, staging, prognostic monitoring and assessment of the effectiveness of treatments for patients with Alzheimer’s disease and other tauopathies and can serve as new targets for the development of new therapies.

Filamin A (FLNA) is a widely expressed actin-binding protein that regulates cell morphology and is particularly expressed in structured cells such as neurons, although expression levels are generally higher in other organs such as lung, kidney and muscle (Human Protein Atlas). FLNA can be phosphorylated at a number of serine, threonine and tyrosine residues in vivo, and the parent protein of approximately 280 kDa is processed to form fragments of approximately 110 kDa and 90 kDa. It has previously been shown that all three major isoforms of FLNA can be phosphorylated at serine 2152 and that the relative intensity of staining in Western blot with a pS2152-specific antibody can differentiate patients with Alzheimer’s disease (AD) from cognitively normal controls.

Modification of FLNA by proteolytic cleavage and phosphorylation is thought to promote changes in the protein structure and/or conformation in the brain. Other disease-specific factors, yet to be identified, may also render FLNA to have altered conformation(s). FLNA, having an altered conformation, is believed to play a role in a toxic signalling mechanism of AB oligomers. In Alzheimer’s patients, the altered FLNA independently interacts with both the α7-nicotinic acetylcholine receptor (α7nAChR) and toll-like receptor 4 (TLR4). Signalling through a7nAChR is reported to hyperphosphorylate tau and signalling through TLR4 is reported to induce neuroinflammation.

In addition, FLNA is a regulator of the actin cytoskeleton, which is important for synaptic function, suggesting another mechanism through which FLNA can contribute to cognitive dysfunction. As such, altered FLNA represents a promising target for new AD therapeutics and is the target of simufilam (PTI-125), a small molecule structural modulator that lessens or prevents the pathological effects of altered FLNA.

The use of a pS2152 specific antibody for diagnosis of AD has not yet been validated. In part, this may be due to the potential for the antibody to bind to the unphosphorylated protein, or may reflect the lack of consistency in phosphorylation in human populations. Further to the use of pS2152 antibodies in a Western Blot, where individual isoforms can be easily distinguished on the basis of their molecular weights, the ability to distinguish FLNA isoforms (in which the pS2152 epitope is present in each isoform) in a liquid phase assay such as an ELISA remains a challenge. Furthermore, the widespread expression of FLNA, especially that found in platelets, makes measurement in peripheral fluids prone to potential false-positives. To date, it has not been demonstrated that measuring specific FLNA isoforms or phosphorylations can be used reliably for diagnosis of AD.

In view of the limitations of FLNA profiling using antibodies, the present disclosure provides a more specific method of bottom-up mass spectrometry to profile the distribution of FLNA-derived tryptic peptides in the blood plasma of patients with a particular focus on measurement of specific phosphorylation events. Specifically, in some aspects the disclosure provides assays that use the TMTcalibrator™ method (U.S. Pat. No. 10,976,321; [Russel et al., Rapid Commun. Mass Spectrom. 31:153—159 (2017)] where isobarically-labelled digests of AD brain tissue lysate are mixed with similarly-labelled plasma samples and wherein the brain lysate digests are present in different concentrations, and their total is in an excess relative to the total concentration of the digests from the plasma channels. By this arrangement, the high concentration of disease-associated proteins in the brain lysates act as a booster for the same proteins present in the plasma, even when these are at concentrations normally below that required for bottom-up mass spectrometry. Using this approach greatly enhances the ability to measure peptides derived from FLNA, and also allows analysis of a much wider group of proteins with common expression in AD brain and plasma.

TABLE 1

Biomarkers of Group A (Significantly regulated proteins)

| Gene Name | Protein Name | SEQ ID NO: | log2 Fold-Change AD vs Control | pValue | Adjusted pValue |
|---|---|---|---|---|---|
| ARPC1B | Actin-related protein ⅔ complex subunit 1B | 16 | 2.54 | 1.16E-09 | 4.06E-07 |
| ARPC5 | Actin-related protein ⅔ complex subunit 5 | 17 | 1.38 | 4.33E-09 | 1.33E-06 |
| ADK | Adenosine kinase | 18 | 2.10 | 3.95E-06 | 2.43E-04 |
| ANXA3 | Annexin A3 | 19 | 0.98 | 3.15E-08 | 6.55E-06 |
| BIN2 | Bridging integrator 2 | 20 | 4.22 | 9.84E-12 | 4.04E-08 |
| CALD1 | Caldesmon | 21 | 1.83 | 1.54E-08 | 3.79E-06 |
| CALM3 | Calmodulin-3 | 22 | 1.12 | 4.60E-07 | 4.59E-05 |
| CALR | Calreticulin | 23 | 1.22 | 1.03E-07 | 1.39E-05 |
| CAVIN2 | Caveolae-associated protein 2 | 24 | 2.04 | 7.05E-09 | 1.97E-06 |
| CLIC1 | Chloride intracellular channel protein 1 | 25 | 2.09 | 1.24E-07 | 1.63E-05 |
| MOXD1 | DBH-like monooxygenase protein 1 | 26 | 1.45 | 3.31E-06 | 2.17E-04 |
| DAP | Death-associated protein 1 | 27 | 2.06 | 3.89E-06 | 2.43E-04 |
| ERLIN1 | Erlin-1 | 28 | 0.97 | 1.58E-06 | 1.27E-04 |
| FLNA | Filamin-A | 1 | 1.73 | 1.36E-10 | 1.03E-07 |
| PDE8B | High affinity cAMP-specific and IBMX-insensitive 3’,5’-cyclic phosphodiesterase 8B | 29 | -1.11 | 6.60E-08 | 1.03E-05 |
| IHH | Indian hedgehog protein | 30 | -0.97 | 1.06E-06 | 9.42E-05 |
| ITGA2B | Integrin alpha-IIb | 31 | 4.41 | 7.13E-08 | 1.07E-05 |
| ITGB3 | Integrin beta-3 | 32 | 3.76 | 2.39E-08 | 5.26E-06 |
| LRRFIP2 | Leucine-rich repeat flightless-interacting protein 2 | 33 | 1.26 | 2.03E-06 | 1.47E-04 |
| SERPINB1 | Leukocyte elastase inhibitor | 34 | 1.64 | 4.38E-08 | 7.65E-06 |
| CTSA | Lysosomal protective protein | 35 | 1.24 | 1.51E-06 | 1.24E-04 |
| MANF | Mesencephalic astrocyte-derived neurotrophic factor | 36 | 1.71 | 9.32E-08 | 1.35E-05 |
| MSN | Moesin | 37 | 1.50 | 1.03E-09 | 3.92E-07 |
| MYL6 | Myosin light polypeptide 6 | 38 | 2.61 | 5.90E-10 | 2.47E-07 |
| MTPN | Myotrophin | 39 | 1.11 | 4.45E-09 | 1.33E-06 |
| PDLIM1 | PDZ and LIM domain protein 1 | 40 | 2.76 | 3.47E-10 | 1.62E-07 |
| PPL | Periplakin | 41 | 1.04 | 2.66E-06 | 1.80E-04 |
| PLEK | Pleckstrin | 42 | 3.17 | 1.73E-10 | 1.03E-07 |
| PFN1 | Profilin-1 | 43 | 1.35 | 1.65E-10 | 1.03E-07 |
| PDIA3 | Protein disulfide-isomerase A3 | 44 | 0.98 | 1.91E-06 | 1.43E-04 |
| S100A12 | Protein S100-A12 | 45 | 3.68 | 2.10E-07 | 2.38E-05 |
| S100A4 | Protein S100-A4 | 46 | 3.20 | 5.70E-07 | 5.31E-05 |
| S100A9 | Protein S100-A9 | 47 | 2.74 | 2.26E-06 | 1.58E-04 |
| ARHGDIB | Rho GDP-dissociation inhibitor 2 | 48 | 1.05 | 1.84E-06 | 1.41E-04 |
| SH3BGRL3 | SH3 domain-binding glutamic acid-rich-like protein 3 | 49 | 2.09 | 1.90E-08 | 4.42E-06 |
| STOM | Stomatin | 50 | 1.46 | 3.28E-08 | 6.55E-06 |
| TLN1 | Talin-1 | 51 | 1.54 | 1.72E-10 | 1.03E-07 |
| TMSB4X | Thymosin beta-4 | 52 | 2.10 | 4.35E-08 | 7.65E-06 |
| TAGLN2 | Transgelin-2 | 53 | 2.55 | 1.33E-10 | 1.03E-07 |
| TPM3 | Tropomyosin alpha-3 chain | 54 | 1.33 | 1.88E-07 | 2.25E-05 |
| TPM4 | Tropomyosin alpha-4 chain | 55 | 2.92 | 1.93E-11 | 4.04E-08 |
| LYN | Tyrosine-protein kinase Lyn | 56 | 1.02 | 1.60E-06 | 1.27E-04 |
| VASP | Vasodilator-stimulated phosphoprotein | 57 | 2.79 | 3.73E-08 | 7.11 E-06 |
| WDR1 | WD repeat-containing protein 1 | 58 | 1.57 | 2.17E-10 | 1.14E-07 |
| ZYX | Zyxin | 59 | 1.84 | 9.71E-08 | 1.36E-05 |

TABLE 2

Biomarkers of Group B (Significantly regulated peptides)

| Protein Name | Uniprot Accession No. | Peptide Sequence | SEQ ID NO: | log2 Fold-Change AD vs Control | pValue | Adjusted pValue |
|---|---|---|---|---|---|---|
| Actin-related protein ⅔ complex subunit 1B | 015143 | sLESALk | 60 | 2.41 | 2.46E-10 | 3.91E-07 |
| Alpha-enolase | P06733 | aVEHINk | 61 | 3.58 | 1.44E-10 | 2.71E-07 |
| Bridging integrator 2 | Q9UBW5 | aSLGTGTASPR | 62 | 4.22 | 7.71E-12 | 6.12E-08 |
| Bridging integrator 2 | Q9UBW5 | aTASPRPSSGNIPS [p] sPTASGG G[p]sPTSPR | 63 | 4.32 | 8.25E-13 | 1.64E-08 |
| Caldesmon | Q05682 | gS [p] sLkIEER | 64 | 5.86 | 6.82E-12 | 6.12E-08 |
| Caveolae-associated protein 2 | 095810 | gIQNDLTk | 65 | 2.73 | 8.54E-10 | 7.65E-07 |
| Chloride intracellular channel protein 1 | 000299 | nSNPALNDNLEk | 66 | 4.02 | 8.47E-11 | 2.24E-07 |
| Claudin-5 | 000501 | rP[p]tATGDYDkk | 67 | 4.94 | 2.85E-13 | 1.13E-08 |
| Endoplasmic reticulum chaperone BiP | P11021 | 1YGSAGPPPTGEEDTAEkDEL | 68 | 3.09 | 8.67E-10 | 7.65E-07 |
| Filamin-A | P21333 | aEISFEDRk | 69 | 3.24 | 8.39E-10 | 7.65E-07 |
| Filamin-A | P21333 | sSFTVDcSk | 70 | 3.93 | 5.28E-10 | 5.98E-07 |
| Filamin-A | P21333 | tPcEEILVk | 71 | 3.46 | 3.44E-10 | 4.57E-07 |
| Junctional adhesion molecule A | Q9Y624 | kVIYSQP[p]sAR | 72 | 6.62 | 1.88E-11 | 1.07E-07 |
| Leucine-rich repeat flightless-interacting protein 2 | Q9Y608 | nSASATTPL [p] sGNSSR | 73 | 3.07 | 5.22E-11 | 1.72E-07 |
| LIM and SH3 domain protein 1 | Q14847 | tQDQISNIk | 74 | 2.64 | 7.06E-10 | 7.18E-07 |
| Linker for activation of T-cells family member 1 | 043561 | [p] sQPLGGSHR | 75 | 4.50 | 1.02E-10 | 2.39E-07 |
| Moesin | P26038 | aQQELEEQTRR | 76 | 2.34 | 5.81E-10 | 6.23E-07 |
| Myosin light polypeptide 6 | P60660 | s DEMNVk | 77 | 3.80 | 1.13E-11 | 7.47E-08 |
| PDZ and LIM domain protein 1 | 000151 | gcTDNLTLTVAR | 78 | 2.76 | 3.49E-10 | 4.57E-07 |
| Platelet endothelial aggregation receptor 1 | Q5VY43 | hPP [p] sPPLR | 79 | 4.21 | 6.00E-11 | 1.83E-07 |
| Pleckstrin | P08567 | gSTLTSPcQDFGkR | 80 | 3.17 | 1.51E-10 | 2.72E-07 |
| Profilin-1 | P07737 | eGVHGGLINkk | 81 | 2.53 | 4.50E-10 | 5.25E-07 |
| Profilin-1 | P07737 | sTGGAPTFNVTVTk | 82 | 2.70 | 3.20E-10 | 4.57E-07 |
| Protein phosphatase 1 regulatory subunit 12A | O14974 | k[p]tGSYGALAEITASk | 83 | 3.77 | 8.10E-10 | 7.65E-07 |
| Purine nucleoside phosphorylase | P00491 | aNHEEVLAAGk | 84 | 3.01 | 7.94E-10 | 7.65E-07 |
| SH3 domain-binding glutamic acid-rich-like protein 3 | Q9H299 | iQYQLVDISQDNALRDEMR | 85 | 2.34 | 6.05E-10 | 6.31E-07 |
| SH3 domain-binding glutamic acid-rich-like protein 3 | Q9H299 | vYSTSVTGSR | 86 | 2.04 | 3.57E-10 | 4.57E-07 |
| Src substrate cortactin | Q14247 | akTQ[p]tPPV[p]sPAPQPTEER | 87 | 3.82 | 3.93E-12 | 5.20E-08 |
| Talin-1 | Q9Y490 | aLDGAFTEENR | 88 | 3.12 | 4.47E-10 | 5.25E-07 |
| Talin-1 | Q9Y490 | tIMVDDSk | 89 | 2.19 | 3.92E-10 | 4.86E-07 |
| Transgelin-2 | P37802 | gPAYGLSR | 90 | 4.44 | 5.06E-11 | 1.72E-07 |
| Transgelin-2 | P37802 | nMAcVQR | 91 | 3.98 | 3.40E-10 | 4.57E-07 |
| Tropomyosin alpha-1 chain | P09493 | aQkDEEkMEIQEIQLk | 92 | 2.94 | 1.32E-10 | 2.61E-07 |
| Tropomyosin alpha-4 chain | P67936 | aEFAER | 93 | 4.54 | 9.85E-11 | 2.39E-07 |
| Tropomyosin alpha-4 chain | P67936 | lATALQk | 94 | 4.19 | 1.12E-10 | 2.40E-07 |
| Tropomyosin alpha-4 chain | P67936 | aEVSELk | 95 | 4.37 | 7.28E-11 | 2.06E-07 |
| Tropomyosin alpha-4 chain | P67936 | ekAEGDVAALNR | 96 | 4.99 | 3.09E-10 | 4.57E-07 |
| Tropomyosin alpha-4 chain | P67936 | kIQALQQQADEADER | 97 | 3.80 | 2.58E-11 | 1.25E-07 |
| Tropomyosin alpha-4 chain | P67936 | sLEAASEk | 98 | 5.18 | 2.82E-11 | 1.25E-07 |
| Tropomyosin alpha-4 chain | P67936 | tIDDLEEk | 99 | 5.75 | 4.88E-11 | 1.72E-07 |
| UV excision repair protein RAD23 homolog B | P54727 | nQPQFQQMR | 100 | 2.76 | 2.02E-10 | 3.49E-07 |
| WD repeat-containing protein 1 | 075083 | lYSILGTTLkDEGk | 101 | 2.08 | 2.27E-10 | 3.75E-07 |
| WD repeat-containing protein 1 | O75083 | vINSVDIk | 102 | 2.12 | 8.08E-10 | 7.65E-07 |
| WD repeat-containing protein 1 | 075083 | yEYQPFAGk | 103 | 2.25 | 1.15E-10 | 2.40E-07 |
| Zyxin | Q15942 | gPPASSPAPAPkF[p]sPVTPk | 104 | 3.78 | 5.70E-10 | 6.23E-07 |

TABLE 3

Biomarkers of Group C (Additional Phosphopeptides)

| Protein Name | Uniprot Accession No. | Peptide Sequence | SEQ ID NO: | log2 Fold-Change AD vs Control | pValue | Adjusted pValue |
|---|---|---|---|---|---|---|
| Claudin-5 | O00501 | rP [p ]tATGDYDkk | 105 | 4.94 | 3.07E-13 | 2.87E-09 |
| Protein phosphatase 1 regulatory subunit 12A | O14974 | k [p] tGSYGALAEITASk | 106 | 3.77 | 4.37E-10 | 3.75E-07 |
| Linker for activation of T-cells family member 1 | 043561 | [p] sPQPLGGSHR | 107 | 4.50 | 5.19E-11 | 6.92E-08 |
| Caveolae-associated protein 2 | 095810 | kVD [p] sLkk | 108 | 6.10 | 4.26E-09 | 1.99E-06 |
| Platelet glycoprotein Ib alpha chain | P07359 | ySGH [p] sL | 109 | 6.42 | 6.24E-10 | 4.16E-07 |
| Platelet glycoprotein Ib beta chain | P13224 | l [p] sLTDPLVAER | 110 | 4.70 | 1.00E-09 | 6.24E-07 |
| Filamin-A | P21333; 075369 | vkE [p] sITR | 111 | 5.92 | 1.57E-09 | 8.76E-07 |
| Transgelin-2 | P37802 | nF [p] sDNQLQEGk | 112 | 2.44 | 1.59E-09 | 8.76E-07 |
| Caldesmon | Q05682 | gS [p] sLkIEER | 113 | 5.86 | 3.16E-12 | 9.85E-09 |
| Nexilin | Q0ZGT2 | eMLA [p] sDDEEDVSSkVEk | 114 | 4.98 | 4.42E-10 | 3.75E-07 |
| Src substrate cortactin | Q14247 | akTQ [p] tPPV [p] sPAPQPTEER | 115 | 3.82 | 5.39E-12 | 1.15E-08 |
| Ras GTPase-activating protein 3 | Q14644 | yG [p] sQEHPIGDk | 116 | 4.07 | 4.25E-09 | 1.99E-06 |
| Eukaryotic translation initiation factor 4H | Q15056 | aYSSFGGGRG [p] sR | 117 | 3.05 | 1.73E-09 | 8.95E-07 |
| Lethal(2) giant larvae protein homolog 1 | Q15334 | iRE [p] sPk | 118 | 2.78 | 4.93E-09 | 2.19E-06 |
| Zyxin | Q15942 | gPPASSPAPAPkF [p] sPVTPk | 119 | 3.78 | 3.39E-10 | 3.51E-07 |
| Zyxin | Q15942 | [p] sPGAPGPLTLk | 120 | 3.96 | 5.43E-10 | 3.90E-07 |
| Platelet endothelial aggregation receptor 1 | Q5VY43 | hPP [p] sPPLR | 121 | 4.21 | 3.75E-11 | 5.83E-08 |
| Synaptotagmin-like protein 4 | Q96C24 | rD [p] sLDkSGLFPEWk | 122 | 4.36 | 5.21E-10 | 3.90E-07 |
| Bridging integrator 2 | Q9UBW5 | aTASPRPSSGNIPS [p] sPTASGG G [p] sPTSPR | 123 | 4.32 | 8.37E-13 | 3.91E-09 |
| Leucine-rich repeat flightless-interacting protein 2 | Q9Y608 | nSASATTPL [p] sGNSSR | 124 | 3.07 | 8.07E-11 | 9.41E-08 |
| Junctional adhesion molecule A | Q9Y624 | kVIYSQP [p] sAR | 125 | 6.62 | 6.15E-12 | 1.15E-08 |

TABLE 4A

Filamin A peptides detected by TMTcalibrator™ mass spectrometry

| FLNA Peptide | SEQ ID NO: | Relative Expression Controls | | | | | |
|---|---|---|---|---|---|---|---|
| | | Old | Old | Young | Young | Young | Old |
| aEAGVPAEFSIWTR | 126 | -0.58 | -0.57 | -0.27 | 0.28 | -0.59 | -0.50 |
| aEGPGLSR | 127 | -0.93 | -1.48 | -1.08 | -1.18 | -0.89 | -1.34 |
| vkE[p]sITR | 128 | -0.66 | 0.42 | 0.28 | -0.22 | 0.87 | -3.28 |
| aEISFEDRk | 129 | -1.54 | -1.29 | -1.79 | -1.11 | -1.46 | -1.52 |
| aFGPGLQGGSAGSPAR | 130 | -0.15 | -0.46 | 0.06 | 0.32 | -0.30 | -0.59 |
| aGGPGLER | 131 | -1.77 | -1.17 | -1.97 | -2.91 | -1.92 | -0.70 |
| aGQSAAGAAPGGGVDTR | 132 | 0.37 | 1.10 | ND | 0.39 | 0.46 | 1.25 |
| aGVAPLQVk | 133 | -0.62 | -0.53 | 0.13 | 0.07 | -0.31 | -0.38 |
| aNLPQSFQVDTSk | 134 | 0.76 | 0.23 | 0.80 | 1.40 | 1.16 | 1.07 |
| aSGPGLNTTGVPASLPVEFTIDAk | 135 | 2.85 | 2.44 | 3.06 | 2.86 | 2.55 | 2.53 |
| aTcAPQHGAPGPGPADASk | 136 | -4.90 | -4.73 | -7.14 | -2.99 | -2.65 | -3.04 |
| aWGPGLEGGVVGk | 137 | 0.68 | 0.41 | 0.17 | 0.80 | 0.54 | 0.42 |
| aP[p]sVANVGSHcDLSLk | 138 | ND | ND | ND | -2.66 | ND | ND |
| aYGPGIEPTGNMVk | 139 | -1.04 | -0.98 | -0.31 | 0.05 | -1.03 | -1.13 |
| cSGPGLER | 140 | -3.00 | -2.90 | -2.04 | -2.35 | -1.86 | -1.60 |
| cSGPGLSPGMVR | 141 | -1.15 | -1.61 | -0.43 | 0.03 | -0.66 | -0.13 |
| dAGEGGLSLAIEGPSk | 142 | -0.87 | -1.66 | -1.37 | -0.56 | -0.29 | -0.98 |
| dAGEGLLAVQITDPEGkPk | 143 | -0.43 | -0.47 | -0.67 | -0.28 | -0.17 | -0.41 |
| dAPQDFHPDR | 144 | -0.41 | ND | ND | ND | -0.59 | -1.03 |
| cSGPGL[p]sPGMVR | 145 | -2.43 | -2.29 | -2.90 | -2.80 | -2.93 | -0.76 |
| dGScGVAYVVQEPGDYEVSVk | 146 | 1.17 | 1.02 | 0.62 | 0.97 | 0.75 | 0.59 |
| dLAEDAPWk | 147 | 0.01 | -0.02 | -0.16 | 0.53 | -0.19 | -0.06 |
| dVDIIDHHDNTYTVk | 148 | -3.11 | -2.85 | ND | -3.37 | -2.67 | -3.38 |
| eAGAGGLAIAVEGPSk | 149 | 0.81 | 0.45 | 0.75 | 1.04 | 1.10 | 0.74 |
| eEPcLLkR | 150 | -1.73 | -0.81 | -0.68 | -0.67 | -0.74 | -0.93 |
| eGPYSISVLYGDEEVPR | 151 | ND | 1.12 | 1.50 | 1.35 | 0.94 | ND |
| eNGVYLIDVk | 152 | -1.03 | -1.04 | -1.18 | -0.29 | -0.81 | -1.12 |
| eTGEHLVHVk | 153 | -2.33 | -1.72 | -1.86 | -1.78 | -1.28 | -1.46 |
| fNEEHIPDSPFVVPVASPSGDAR | 154 | 0.07 | 0.01 | 0.42 | 0.54 | -0.22 | 0.24 |
| fTVETR | 155 | -1.10 | -0.99 | -0.80 | -0.60 | -0.63 | -0.57 |
| gAGTGGLGLAVPSEAk | 156 | 1.78 | 1.80 | 1.59 | 1.93 | 1.86 | 1.51 |
| gLVEPVDVVDNADGTQTVNYVPSR | 157 | 0.02 | -0.49 | -2.34 | 0.62 | 0.75 | -2.43 |
| gTVEPQLEAR | 158 | -0.46 | 0.32 | 0.22 | -0.12 | -0.01 | -0.38 |
| iEcDDkGDGScDVR | 159 | -2.11 | -2.80 | -2.80 | -1.44 | -1.51 | -1.90 |
| iPEISIQDMTAQV[?p]t[?p]sPSGk | 160 | 0.75 | 0.45 | 0.14 | 1.07 | 0.74 | 0.93 |
| iVGPSGAAVPck | 161 | 2.04 | 1.46 | 0.94 | 2.22 | 1.66 | 1.88 |
| kGEITGEVR | 162 | -1.69 | -1.31 | -1.34 | -0.80 | -1.21 | -1.42 |
| lQVEPAVDTGVQcYGPGIEGQGVFR | 163 | -0.56 | ND | -0.62 | 0.18 | 0.38 | -0.74 |
| mDcQEcPEGYR | 164 | ND | ND | 0.82 | -0.29 | 1.31 | ND |
| mScMDNk | 165 | -0.83 | -0.20 | 0.50 | -0.29 | -0.13 | -1.06 |
| rAP[p]sVANVGSHcDLSLk | 166 | ND | -1.99 | -0.99 | -0.48 | -1.63 | -2.08 |
| sAGQGEVLVYVEDPAGHQEEAk | 167 | 2.23 | 2.21 | 2.75 | 0.73 | 1.88 | 1.96 |
| sPFEVYVDk | 168 | -0.28 | -0.24 | 0.11 | -0.30 | -0.48 | -0.22 |
| sPFSVAVSPSLDLSk | 169 | 2.28 | 2.21 | 2.23 | 2.47 | 2.08 | 2.12 |
| sPYTVTVGQAcNPSAcR | 170 | ND | ND | 1.47 | ND | ND | ND |
| sSFTVDcSk | 171 | -1.41 | -1.36 | -1.85 | -0.52 | -0.79 | -0.75 |
| tFSVWYVPEVTGTHk | 172 | 0.12 | 0.06 | 0.41 | 0.48 | 0.17 | 0.62 |
| tGVAVNkPAEFTVDAk | 173 | 0.11 | -0.52 | 0.12 | 0.32 | 0.08 | 0.78 |
| tHEAEIVEGENHTYcIR | 174 | ND | ND | ND | ND | 2.33 | ND |
| tPcEEILVk | 175 | -0.87 | -0.84 | -0.62 | -0.61 | -0.78 | -1.03 |
| vANPSGNLTETYVQDR | 176 | -0.55 | -0.20 | -0.99 | -0.98 | -0.35 | -0.24 |
| vAQPTITDNkDGTVTVR | 177 | -2.22 | -1.89 | -2.24 | -1.76 | -1.71 | -1.60 |
| vDVGkDQEFTVk | 178 | -0.66 | -0.67 | -1.21 | -0.60 | -1.04 | -0.76 |
| vEPGLGADNSVVR | 179 | 0.44 | 0.23 | 0.36 | 1.01 | 0.63 | 0.14 |
| vQVQDNEGcPVEALVk | 180 | 0.01 | 0.03 | 0.03 | -0.39 | 0.13 | -0.05 |
| vTAQGPGLEPSGNIANk | 181 | -1.13 | -1.81 | -1.65 | -0.76 | -0.75 | -1.04 |
| vTVLFAGQHIAk | 182 | 2.53 | 2.33 | 2.78 | 3.23 | 2.63 | 2.48 |
| wGDEHIPGSPYR | 183 | -0.27 | -0.87 | -0.82 | -0.67 | -0.51 | -0.24 |
| yGGDEIPFSPYR | 184 | -0.60 | -0.55 | -1.45 | -0.44 | -0.70 | -0.90 |
| yTPVQQGPVGVNVTYGGDPIPk | 185 | -0.04 | -0.21 | -0.04 | 0.27 | -0.74 | -0.02 |

TABLE 4B

Filamin A peptides detected by TMTcalibrator™ mass spectrometry

| FLNA Peptide | SEQ ID NO: | Relative Expression Alzheimer’s Disease Subjects | | | | | |
|---|---|---|---|---|---|---|---|
| aEAGVPAEFSIWTR | 126 | 1.66 | 1.24 | 0.88 | 0.66 | 1.93 | 1.58 |
| aEGPGLSR | 127 | 1.74 | 1.46 | 1.36 | 0.85 | 2.02 | 1.49 |
| vkE[p]sITR | 128 | 6.01 | 5.42 | 5.24 | 4.89 | 6.08 | 5.92 |
| aEISFEDRk | 129 | 2.07 | 1.72 | 1.46 | 0.99 | 2.19 | 2.01 |
| aFGPGLQGGSAGSPAR | 130 | 2.41 | 1.94 | 1.86 | 1.44 | 2.67 | 2.43 |
| aGGPGLER | 131 | 1.54 | 1.17 | 0.73 | 0.40 | 0.36 | 1.10 |
| aGQSAAGAAPGGGVDTR | 132 | 1.64 | 1.81 | 0.93 | 0.88 | 1.71 | 0.86 |
| aGVAPLQVk | 133 | 0.97 | 0.79 | 0.68 | 0.67 | 0.79 | 0.79 |
| aNLPQSFQVDTSk | 134 | 2.20 | 2.03 | 1.76 | 1.76 | 2.75 | 2.11 |
| aSGPGLNTTGVPASLPVEFTIDAk | 135 | 2.56 | 2.63 | 2.61 | 2.70 | 2.80 | 2.70 |
| aTcAPQHGAPGPGPADASk | 136 | 1.47 | 1.43 | 0.30 | 0.24 | 1.52 | 0.99 |
| aWGPGLEGGVVGk | 137 | 2.00 | 1.48 | 1.36 | 1.18 | 2.14 | 1.73 |
| aP[p]sVANVGSHcDLSLk | 138 | -0.44 | -1.63 | -1.40 | -1.67 | -0.85 | -0.39 |
| aYGPGIEPTGNMVk | 139 | 1.42 | 0.83 | 0.89 | 0.23 | 1.67 | 1.21 |
| cSGPGLER | 140 | -2.26 | -2.19 | -3.33 | -1.89 | -1.03 | -2.66 |
| cSGPGLSPGMVR | 141 | -1.38 | -0.49 | -0.60 | -0.53 | -0.45 | -1.23 |
| dAGEGGLSLAIEGPSk | 142 | 1.34 | 0.94 | 1.06 | 0.31 | 1.14 | 1.28 |
| dAGEGLLAVQITDPEGkPk | 143 | 1.00 | 0.99 | 0.82 | 0.42 | 1.11 | 1.18 |
| dAPQDFHPDR | 144 | 1.68 | 1.12 | 1.52 | 0.95 | 2.50 | 1.78 |
| cSGPGL[p]sPGMVR | 145 | -2.00 | -2.06 | -3.65 | -1.56 | -0.20 | -1.48 |
| dGScGVAYVVQEPGDYEVSVk | 146 | 1.73 | 1.44 | 1.46 | 1.37 | 1.09 | 1.11 |
| dLAEDAPWk | 147 | 0.87 | 0.36 | 0.52 | 0.56 | 0.86 | 0.52 |
| dVDIIDHHDNTYTVk | 148 | 1.81 | 1.30 | 0.77 | 0.52 | 2.17 | 1.61 |
| eAGAGGLAIAVEGPSk | 149 | 2.40 | 1.86 | 2.02 | 1.70 | 2.42 | 2.35 |
| eEPcLLkR | 150 | 1.08 | 0.77 | 1.28 | 0.57 | 2.04 | 0.93 |
| eGPYSISVLYGDEEVPR | 151 | 1.32 | 1.47 | 1.39 | 0.82 | 1.40 | 0.80 |
| eNGVYLIDVk | 152 | 0.47 | 0.49 | 0.04 | -0.05 | 0.11 | 0.31 |
| eTGEHLVHVk | 153 | 2.43 | 1.83 | 2.04 | 1.31 | 2.65 | 2.53 |
| fNEEHIPDSPFVVPVASPSGDAR | 154 | 1.00 | 0.97 | 0.87 | 0.62 | 1.60 | 1.20 |
| fTVETR | 155 | 0.68 | 0.38 | 0.22 | 0.03 | 0.87 | 0.69 |
| gAGTGGLGLAVEGPSEAk | 156 | 3.10 | 2.81 | 2.97 | 2.52 | 3.11 | 2.99 |
| gLVEPVDVVDNADGTQTVNYVPSR | 157 | -2.55 | -2.60 | -3.00 | -2.52 | -1.59 | 0.29 |
| gTVEPQLEAR | 158 | 1.23 | 0.99 | 0.73 | 0.31 | 1.73 | 1.45 |
| iEcDDkGDGScDVR | 159 | 2.15 | 1.62 | 1.26 | 0.89 | 2.50 | 1.64 |
| iPEISIQDMTAQV[?p]t[?p]sPSGk | 160 | 2.97 | 1.33 | 2.81 | 1.61 | 1.48 | 3.83 |
| iVGPSGAAVPck | 161 | 2.32 | 2.34 | 1.99 | 2.37 | 2.66 | 2.20 |
| kGEITGEVR | 162 | 1.05 | 1.01 | 0.99 | 0.38 | 0.58 | 0.72 |
| lQVEPAVDTSGVQcYGPGIEGQGVFR | 163 | 0.78 | 0.38 | 1.00 | 0.71 | 0.91 | 0.89 |
| mDcQEcPEGYR | 164 | 1.54 | 0.84 | 1.20 | 0.67 | 1.96 | -2.59 |
| mScMDNk | 165 | 3.14 | 2.47 | 2.43 | 1.67 | 2.65 | 2.50 |
| rAP[p]sVANVGSHcDLSLk | 166 | -0.96 | -1.80 | -1.80 | -0.55 | -1.04 | -0.68 |
| sAGQGEVLVYVEDPAGHQEEAk | 167 | 1.52 | 1.08 | 0.80 | 1.01 | 1.96 | 1.57 |
| sPFEVYVDk | 168 | 0.75 | 0.30 | 0.27 | 0.12 | 1.09 | 0.48 |
| sPFSVAVSPSLDLSk | 169 | 2.42 | 2.17 | 2.45 | 2.38 | 2.58 | 2.52 |
| sPYTVTVGQAcNPSAcR | 170 | 2.35 | 2.13 | 2.04 | 1.84 | 2.33 | 2.73 |
| sSFTVDcSk | 171 | 3.08 | 2.50 | 2.83 | 1.77 | 3.19 | 3.18 |
| tFSVWYVPEVTGTHk | 172 | 0.61 | 0.49 | 0.66 | 0.67 | 1.24 | 0.73 |
| tGVAVNkPAEFTVDAk | 173 | 1.87 | 1.52 | 1.35 | 1.15 | 1.02 | 1.47 |
| tHEAEIVEGENHTYcIR | 174 | 0.94 | 1.61 | 1.72 | 2.26 | 2.14 | 1.34 |
| tPcEEILVk | 175 | 2.77 | 2.22 | 2.63 | 1.56 | 3.01 | 3.02 |
| vANPSGNLTETYVQDR | 176 | 2.01 | 1.66 | 1.36 | 0.96 | 2.01 | 1.87 |
| vAQPTITDNkDGTVTVR | 177 | 0.38 | 0.06 | 0.29 | -0.40 | 0.55 | -0.03 |
| vDVGkDQEFTVk | 178 | 1.74 | 1.40 | 1.28 | 0.35 | 2.16 | 1.72 |
| vEPGLGADNSVVR | 179 | 2.42 | 1.92 | 1.93 | 1.40 | 1.92 | 2.14 |
| vQVQDNEGcPVEALVk | 180 | 1.74 | 1.30 | 1.31 | 0.58 | 2.00 | 1.52 |
| vTAQGPGLEPSGNIANk | 181 | 1.37 | 1.11 | 0.93 | 0.66 | 1.88 | 1.49 |
| vTVLFAGQHIAk | 182 | 3.10 | 3.11 | 2.85 | 2.93 | 3.28 | 3.37 |
| wGDEHIPGSPYR | 183 | 1.19 | 1.04 | 1.13 | 0.30 | 0.61 | 1.38 |
| yGGDEIPFSPYR | 184 | 0.50 | 0.32 | 0.21 | -0.04 | 0.18 | 0.57 |
| yTPVQQGPVGVNVTYGGDPIPk | 185 | 1.76 | 1.42 | 1.45 | 1.00 | 2.04 | 1.63 |

### EXAMPLES

It should be understood that the above-described embodiments and the following examples are given by way of illustration, not limitation. Various changes and modifications within the scope of the present invention will become apparent to those skilled in the art from the present description.

### Example 1: TMTcalibrator™ Analysis of Histopaque®-1077 Prepared Alzheimer’s Plasma and EDTA Control Plasma

### Samples

Human plasma samples from 6 controls (3 elderly controls, 3 young cognitive intact) were drawn into Vacutainer® tubes (Becton, Dickinson and Company Franklin Lakes, NJ) containing K2EDTA. [Plasma from controls were negative (-) for the 90 kDa FLNA biomarker as revealed by Western blot using a phospho-specific rabbit polyclonal antibody specific for pS2152 (Origene TA313881, Origene Technologies Rockville, MD).] Within 30 minutes after being drawn, the blood was centrifuged blood at approximately 1000 X G for 15 min, preferably at 4-5° C. Within 30 min of centrifuging, plasma was collected. Control plasma was then combined with 5% volume/volume of 20X protease and phosphatase inhibitor cocktails and added, thoroughly mixed by vortexing for 1 minute and aliquoted. The protease-phosphate inhibitor cocktail was prepared by dissolving 1X Roche PhosStop EASYpak together with 1X Roche complete tablets mini EDTA free EASYpack (Thomas Scientific, Swedesboro, NJ) into 500 ml of distilled water. Human plasma samples from 6 AD patients were drawn into Vacutainer® tubes and transported to the laboratory for processing. Human plasma from AD patients were positive (+) for the 90 kDa FLNA biomarker as revealed by Western blot using a phospho-specific rabbit polyclonal antibody specific for pS2152 (Origene TA313881, Origene Technologies Rockville, MD). At the laboratory, 4 ml of the whole blood from the Vacutainer® tube were layered onto 4 ml of Histopaque®-1077 (Sigma-Aldrich) in a 14 ml disposable tube. The disposable tube was centrifuged at 400 g for 30 min at room temperature after which plasma was transferred to 1.5 ml Eppendorf tubes for storage and 5% volume/volume of 20X protease and phosphatase inhibitor cocktails and added, thoroughly mixed by vortexing for 1 minute and aliquoted for use in biomarker assays. Aliquots were stored at -80 C. An aliquot of three post-mortem AD brain (Braak Stage IV-VI) lysates was provided by Proteome Sciences and used as a trigger sample of brain-derived proteins.

### Analytical Method

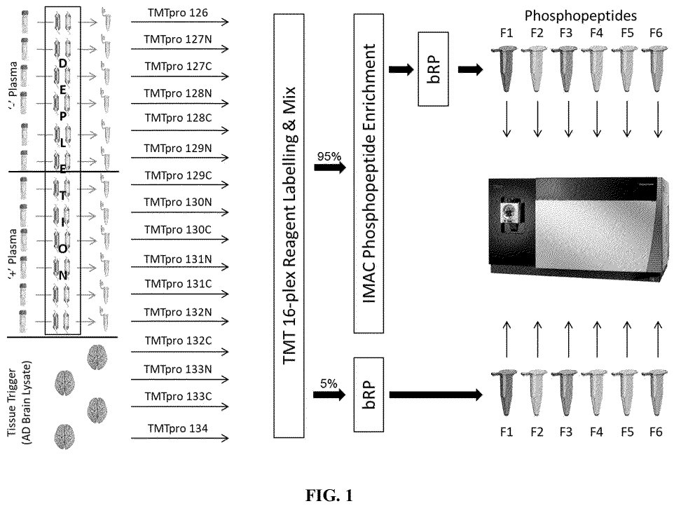

For this experiment, a TMTcalibrator™ phosphoproteomic analysis (FIG. 1) was performed using 12 AD plasma samples with AD brain tissue used as a trigger sample. Plasma samples were depleted from high-abundant proteins using Top14 Abundant Protein Depletion spin columns. Proteins were digested using trypsin and the peptides labelled with TMTpro™ and mixed to generate one TMTpro™ 16-plex sample (reagents available from Thermo Fisher Scientific, Waltham, MA, USA), Four TMTproTM channels were used for the AD brain tissue trigger.

The TMTpro™ 16-plex sample was split into an aliquot for total proteome analysis (non-enriched) and an aliquot for phosphoproteome analysis (phosphopeptide enriched). After phosphopeptide enrichment, 6 phosphopeptide-enriched and 6 non-enriched fractions were generated. Each fraction was subjected to LC-MS2 analysis using a high-performance Orbitrap Fusion™ Tribrid™ mass spectrometer (Thermo Fisher Scientific, Waltham, MA, USA. ) using a data-dependent acquisition method (using an inclusion list for FLNA peptides for the non-enriched fractions). Raw data were searched using Proteome Discoverer™ v2.5 (Thermo Fisher Scientific). Data were further processed using a proprietary bioinformatics pipeline involving filtering, normalisation, biostatistics, annotation and functional analysis. Box-plots of the proteins of interest were also generated.

### Sample Management

Upon receipt, all samples were visually inspected to assess the absence of thawing, correct labelling and general integrity. Samples were stored at -80° C. Details were entered into the Laboratory Information Management System (LIMS) under a unique reference number.

### Brain Lysis

Available protein from pooled AD brain tissue lysates was used as a calibrator/trigger brain sample.

### Protein Concentration Measurement and SDS-PAGE

The protein concentration of plasma, depleted plasma and brain lysate samples were determined by Bradford protein assay and each sample visualised by Coomassie stained (Imperial Stain, Pierce, Thermo Fisher Scientific) SDS-PAGE 4-20% gradient gels (Criterion, Biorad).

### Protein Depletion

35 µL of plasma were depleted using HighSelect Top14 Abundant Protein Depletion (Pierce, Thermo Fisher Scientific) based on the manufacturer’s protocol. For several samples multipe aliquots of 35 µl each were depleted to obtain sufficient amounts (Table S2).

### Digestion and TMTpro Labelling

Calibrator/Trigger brain sample: 6.6 mg of the pooled brain lysate was reduced (dithiothreitol), alkylated (iodoacetamide), digested (trypsin) to generate peptides, desalted (SepPak® tC18 cartridges, Waters Corp., Milford, MA, USA), aliquoted into 4 portions each reflecting a 1:4:6:10 ratio, and lyophilised to dryness.

Analytical depleted plasma samples: 180 µg per individual depleted plasma sample was used. Depleted plasma samples were brought to equal volumes. Samples were reduced, alkylated and digested with trypsin to generate peptides, and after desalting (SepPak^{®} tC18 cartridges) samples were lyophilised to dryness.

Dry peptides (from depleted plasma and brain samples) were dissolved in TEAB/ACN buffer. Peptides were mixed with their respective TMTpro™ reagent (labelling plan shown in Table 2). TMTpro™ labelled samples were treated with hydroxylamine and the labelled digests were pooled to generate the TMTpro™ 16-plex sample containing 12 depleted plasma analytical samples (12 × 180 µg = 2160 µg) + pooled brain digest mixture (about 3780 µg in 4 channels with a ratio of 1:4:6:10) in a protein mass ratio of 1:1.75). 50 µg of the mixtures were purified by solid phase extractions to be used for assessment of labelling efficiency and reporter ion distributions (equimolarity check). Two portions each of 100 µg were taken off as samples for basic-reversed fractionation for the non-enriched arm (including a backup sample) and about 5,690 µg portions for phosphopeptide enrichment.

### Phosphopeptide Enrichment

About 5,690 µg of the TMTpro™ 16-plex sample was enriched for phosphopeptides over two columns of the High Select™ Fe-NTA Phosphopeptide Enrichment Kit (Thermo Scientific Pierce, cat. no. A32992) according to manufacturer’s instructions and combined yielding one pooled phosphopeptide sample.

### Basic Reversed-Phase Fractionation (bRP)

bRP fractionation was conducted for the TMTpro™ 16plex taking A) 100 µg of non-enriched samples and B) enriched phosphopeptides. Here, the Thermo Scientific™ Pierce™ High pH Reversed-Phase Peptide Fractionation Kit (cat. no. 84868) was used according to manufacturer’s instructions.

### Liquid Chromatography Mass Spectrometry (LC-MS/MS)

Each of the individual fractions (6 phosphopeptide enriched and 6 non-enriched fractions per 16plex) was analysed by LC-MS/MS using an EASYnLC-1000 system coupled to an Orbitrap FusionTM TribridTM Mass Spectrometer (both Thermo Scientific). Peptides were resuspended in 2% acetonitrile (ACN) with 0.1% formic acid (FA). From the thermostatted autosampler, samples (about5% of non-enriched and about 25% of phosphopeptide enriched fractions) were loaded onto a 2 cm 75 µm inner diameter (ID) C18 Acclaim PepMap 100 trapping column (Thermo Scientific, PN 164946) and resolved using an increasing gradient of ACN in 0.1% FA through a 50 cm 75 µm ID EasySpray analytical column (Thermo Scientific, PN ES803A) at a flow rate of 200 nL/min. Gradient starting conditions were 8% ACN for non-enriched fractions and 10% ACN for phospho-enriched fractions. The percentage of organic solvent was continuously increased to a maximum of 30 % ACN.

Peptide mass spectra were acquired throughout the entire chromatographic run (180 minutes). The mass spectrometer was operated in data dependent mode with full scans at 120.000 resolution and MS2 fragment scans acquired at 50.000 resolution. The duty cycle was set to 3 seconds, meaning a full scan was acquired at least every 3 seconds, followed by MS2 scans of fragmented precursors picked from the most abundant targets. After being fragmented once, precursors were excluded from additional fragmentation for 30 seconds.

### Computational Mass Spectrometry

In total, 12 separate raw mass spectrometry data files (12 fractions - 6 enriched & 6 non-enriched) were searched in Proteome Discoverer (PD) v2.5 (Thermo Scientific) using the SEQUEST HT Search algorithm. The raw spectra were searched against a uniprot reviewed human database (version January 2021), with full tryptic specificity and up to two missed cleavages. All spectra that were not matched to a peptide with high confidence, were searched again with semi-tryptic specificity to account for potential cleavage products. Spectra still unmatched after the semi-tryptic search, were searched again with no cleavage specificity. TMTpro™ modification of N-termini and lysine residues, as well as carbamidomethylation of cysteine, were set as static modifications; oxidation of methionine was considered as a variable modification. Phosphorylation in serine, threonine and tyrosine residues was set as variable modification in the searches of phosphopeptide enriched fractions only.

The precursor mass tolerance was set to 20 ppm, while the fragment mass tolerance was set to 0.02 Da. The false discovery rate was controlled at 1% on PSM level by the Percolator node incorporated in Proteome Discoverer. The reporter ions quantifier node was set up to extract the raw intensity values for TMTpro™ 16plex mono-isotopic ions (126, 127N, 127C, 128N, 128C, 129N, 129C, 130N, 130C, 131N, 131C, 132N, 132C, 133N, 133C, 134N). All raw reporter ion intensity values were exported to tab delimited text files for further processing and bioinformatic analysis.

### Bioinfonnatics Data Analysis

Statistical analysis was conducted using internally developed software written in R statistical programming language (R Core Team 2017). All data integration tools were developed to work with TMT® labelled MS data and included functionality for dealing with isolation interference [Savitski, M., et al. “Measuring and managing ratio compression for accurate iTRAQ/TMT quantification.” J. Proteome Research 12.8 (2013): 3586-3598], isotopic crosstalk [Rauniyar, N., et al. “Isobaric labeling-based relative quantification in shotgun proteomics.” J. Proteome Research 13.12 (2014): 5293-5309], PSM normalisation, and summarisation into peptides.

### Statistical Analysis

Statistical analysis was conducted using in-house software and was based on Linear Modelling. [T., Tibshirani, et al., The Elements of Statistical Learning: Data Mining, Inference, and Prediction (Vol. 2, pp. 1-758). New York: Springer (2009).] For feature selection moderated t-statistics were selected (Ritchie, M., et al. “Limma Powers Differential Expression Analyses for RNA-sequencing and Microarray Studies.” Nucleic Acids Research 43.7 (2015): e47-e47; Phipson, B., et al. “Robust Hyperparameter Estimation Protects Against Hypervariable Genes and Improves Power to Detect Differential Expression.” Annals of Applied Statistics 10.2 (2016): 946.)], which effectively borrows information from the ensemble of features to aid with inference about each individual feature and is useful for small data sets.

Analysis was performed for 3 different contrasts: Contrast 1 = AD vs Control (old); Contrast 2 = AD vs Control (young); and Contrast 3 = AD vs Control (all). For each contrast, the fold changes, p-values and adjusted p-values (calculated using the Benjamini-Hochberg FDR) are provided. The LIMMA p-values are based on the moderated t-statistics (Ritchie, Phipson) and are the result of information borrowing across all features to overcome the problem of small sample sizes. Multiple testing corrections were applied using the Benjamini-Hochberg procedure.

### Sample Assessment

Protein determination by modified Bradford assay showed that protein concentrations of plasma samples were between 41.9 and 73.6 µg/µl (mean: about 59 µg/µl). AD samples showed a lower mean protein concentration versus controls (48.7 vs 68.5 µg/µl), SDS-PAGE of all plasma samples showed a very homogenous band pattern. Plasma samples showed protein concentrations in the expected range and there were no concerns on sample quality. The lysis of brain tissue pieces delivered sufficient protein for the study. SDS-PAGE of the three brain lysates showed an acceptable band pattern.

### Plasma Depletion

After Top14-depletion of the 12 plasma samples, the total protein masses available were between 189 and 354 µg. SDS-PAGE of Top14 depleted plasma samples showed a homogenous band pattern. Visual assessment confirmed a typical post-depletion pattern with e.g., albumin bands clearly reduced. about95% depletion was achieved.

### TMTpro™ Analytical Quality Control

Analysis of TMTpro™ labelling reaction efficiency of the TMTpro™ 16plex experiment showed that ~98.2% of N-terminal amino groups were labelled, which indicates that labelling was essentially complete. All mass spectrometry runs of the fractionated samples passed internal quality assessments based on the maximum intensity of total ion chromatograms (TICs), number of MS scans and number of peptide spectral matches (PSMs).

### Metrics: Quantified Peptides and Proteins

We report 39,686 peptide sequences with expression values across all 12 patient-derived samples (Table 3). Of these, 9,337 are phosphorylated and associated with combinations of 6,047 distinct, confidently localised phosphosites. The 39,686 peptides are associated with 4,192 distinct protein groups, quantified across all 12 samples. Overall, the application of the TMTcalibrator™ phosphoproteomic workflow to human plasma has provided excellent coverage of the plasma proteome and phosphoproteome.

TABLE 5

Numbers of detected and quantified features

| | Detected | | | Quantified |
|---|---|---|---|---|
| | Total # unique | TMT Plex | All samples | All samples |
| | | ≥1 analytical sample | all analytical samples | all analytical samples |
| Peptides | 46,346 | 46,304 | 35,309 | 39,686 |
| (Phosphopeptides) | (12,525) | (12,504) | (7,118) | (9,337) |
| Phosphosites | 7,578 | 7,578 | 4,763 | 6,047 |
| Protein groups | 4,553 | 4,552 | 3,972 | 4,192 |

In Table 5, the Peptides row represents both non-phosphorylated and phosphorylated peptides, with the latter described in the subsequent row (italicised). Note that Quantified features allows for a certain number of missing values, which are imputed during data pre-processing.

### Feature Selection: Phosphopeptides

We saw a strong up-regulation of phosphopeptides in the AD group compared to controls, with many phosphopeptides associated with expression changes greater than 6-fold. At the applied thresholds, no significantly down-regulated phosphopeptides were detected.

The most highly regulated phosphopeptide was the peptide kVIYSQP[p]sAR (logFC:6.6, Contrast #1) from Junctional adhesion molecule A (F11R, also known as JAM-1), which contains a modification on serine 284. The next most highly regulated phosphopeptide was kVD[p]sLkk (logFC:6.5, Contrast #1) from Caveolae-associated protein 2 (CAVIN2, also known as SDPR), which was modified on Serine 241. The third most highly regulated phosphopeptide was vkE[p]sITR (logFC: 6.4, Contrast #1) - a sequence shared between Filamin A (FLNA) and Filamin-B (FLNB), and modified on Serine 2143 (FLNA) and Serine 2098 (FLNB).

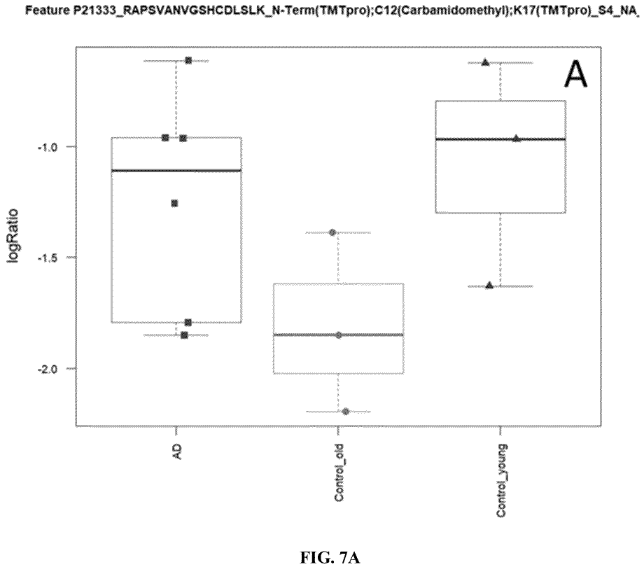

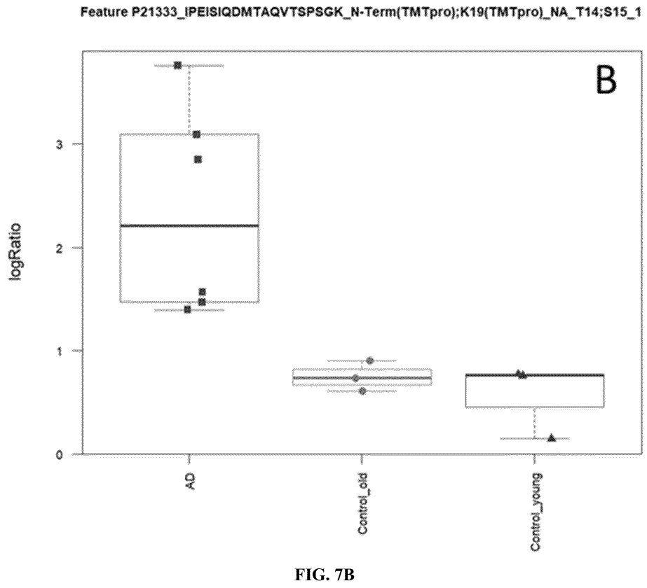

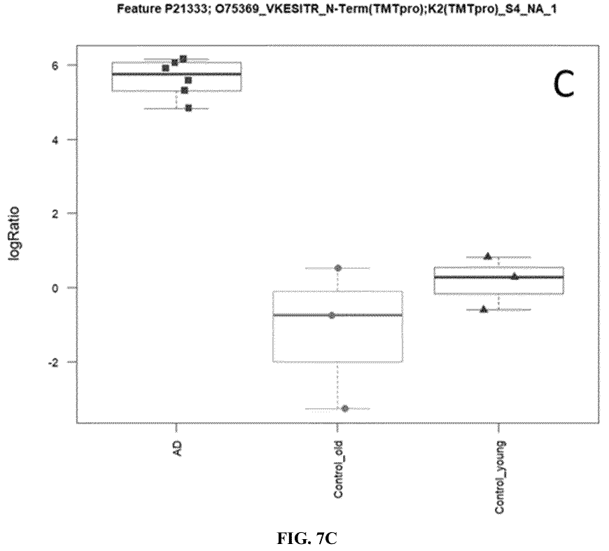

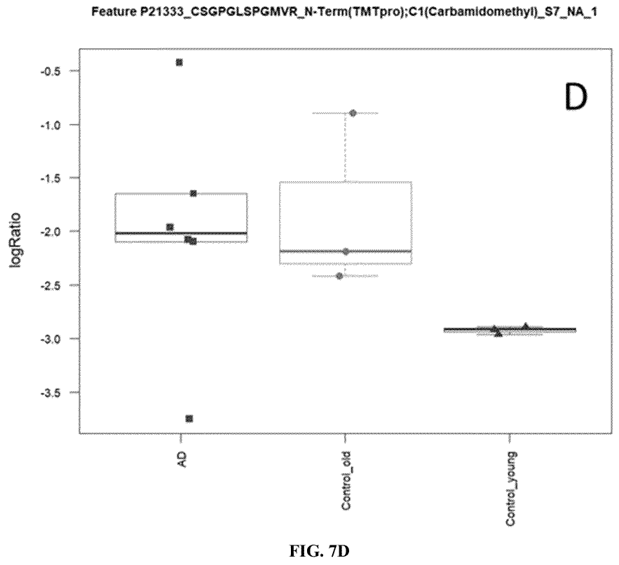

Looking at the four FLNA phosphopeptide features quantified (FIGS. 7A, 7B, 7C, and 7D), phosphorylated FLNA appears to be more highly abundant in AD compared to the total control group. However, there are some differences in expression between and the young and elderly controls. The “RAPSVAN” peptide containing pS2152 is seen at similar levels in AD and young controls but much lower in cognitively healthy elderly controls. Conversely, peptide “CSGPG” containing pS1459 is high in AD and elderly controls and much lower in the young controls.

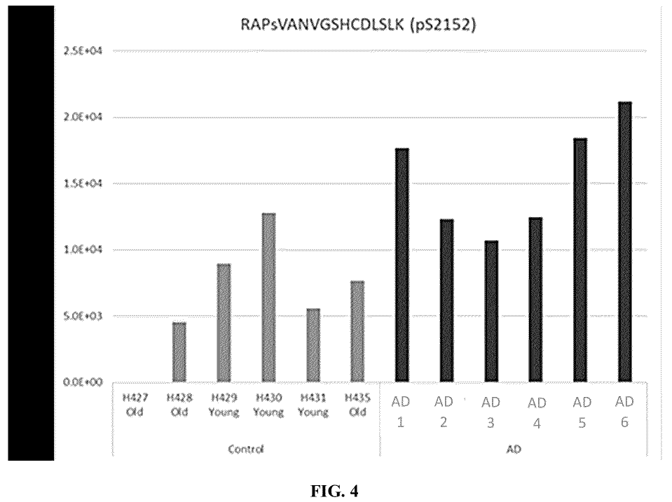

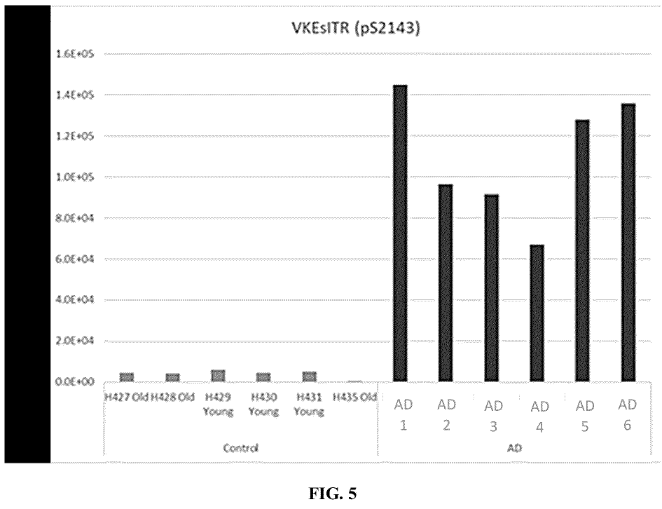

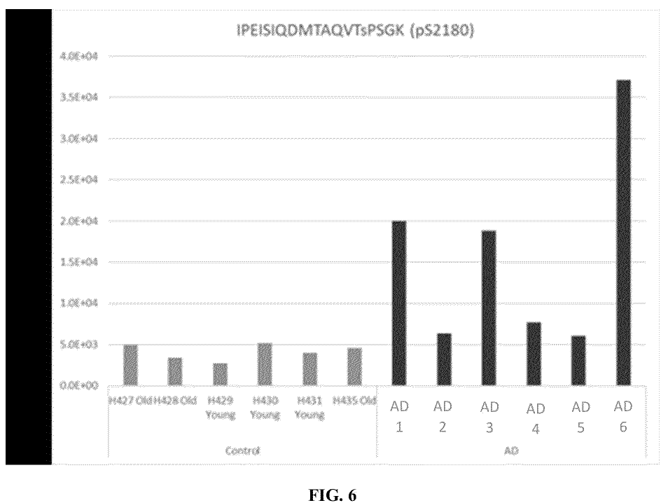

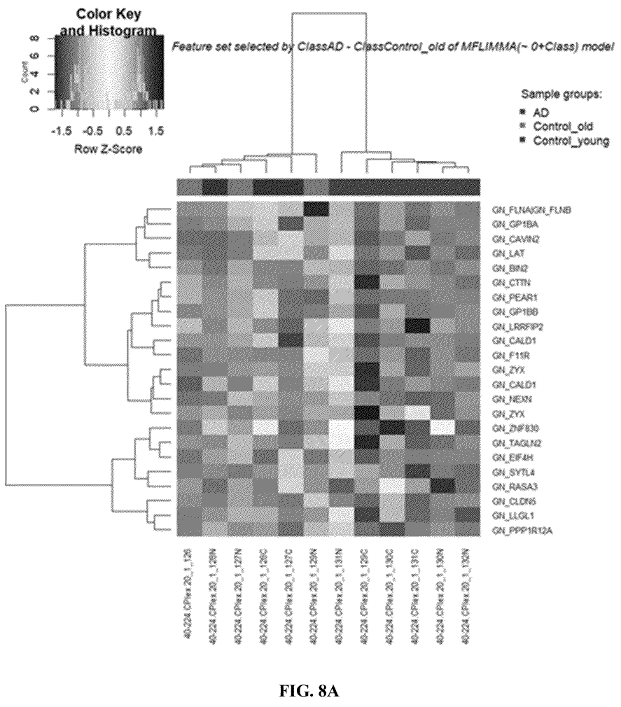

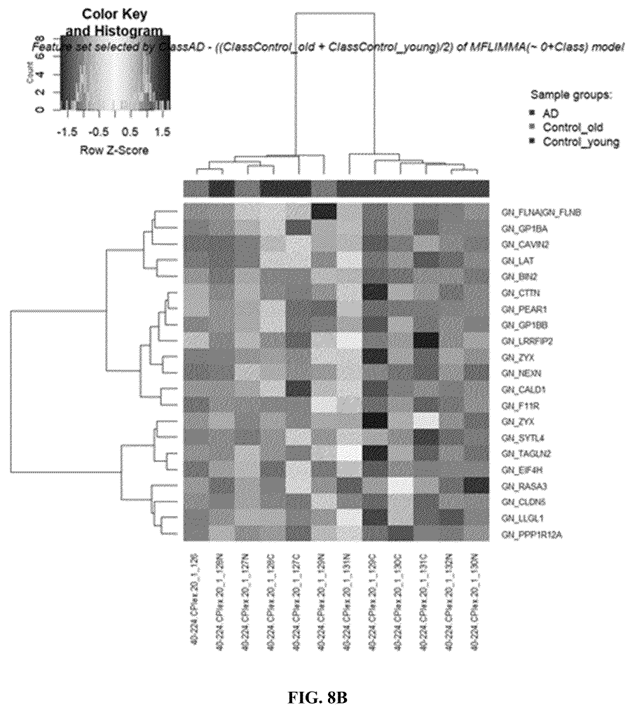

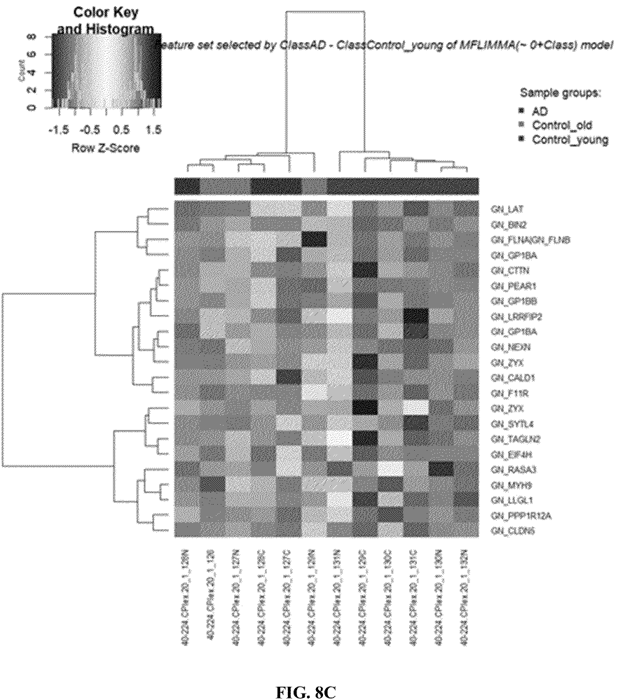

We also performed post hoc analyses to determine whether phosphopeptides selected by their significance of expression changes could explain differences between experimental classes (FIGS. 8A, 8B, and 8C). For all comparisons, selected phosphopeptides were able to successfully distinguish AD and Control groups, indicating that their inter-group expression differences accounted for about 95% (PC1) of the total variance in the dataset. Based on these results, this set of peptides was not associated with significant differences between the two control groups (PC2 < 2%). These results were reflected in the heatmaps, where control groups were mixed on the sample-clustering dendrograms (FIG. 4), while AD groups formed a clear sub-cluster, with significantly higher expression values.

### Feature Selection: Proteins

All contrasts revealed a strong up-regulation of proteins in the disease group compared to controls, with many proteins associated with expression changes greater than 4-fold. At the applied thresholds, only two significantly down-regulated proteins were detected.

Across all three contrasts, we observed a good degree of consistency among proteins, both in terms of their identities and fold change magnitude. The most highly regulated protein was Bridging integrator 2 (BIN2) (logFC:4.5, Contrast #1). The next most highly regulated protein was Integrin alpha-IIb (lTGA2B) (logFC:4.5, Contrast #1). The third most highly regulated protein was Protein S100-A12 (S100A12) (logFC: 3.6, Contrast #1). Of the three highly-regulated phosphopepetides described previously, the corresponding total protein expression (FLNA and CAVIN2) was associated with significantly increased levels in the disease samples (FLNA logFC: 1.8, Contrast #1; CAVIN2 logFC: 1.9, Contrast #1).

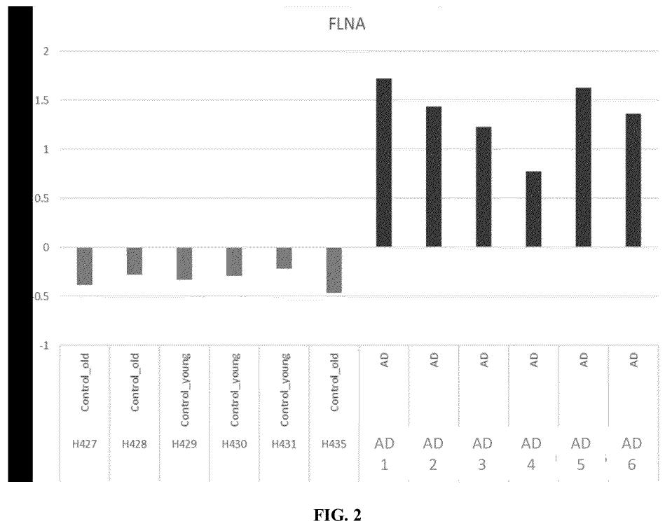

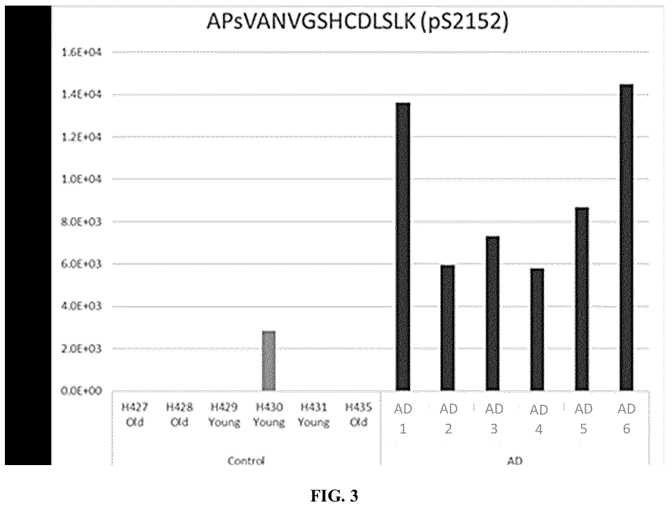

A closer look at the expression profiles between the three observed sample groups for the protein of interest FLNA (FIG. 2) revealed the same trend at the protein level as previously observed at the phosphopeptide level: FLNA expression was higher in the AD group compared to the controls. The comparison of the two control groups in itself showed similar expression with a slightly higher mean for the Young Cognitive Intact group compared to the Elderly Control group.

### Summary of FLNA Coverage

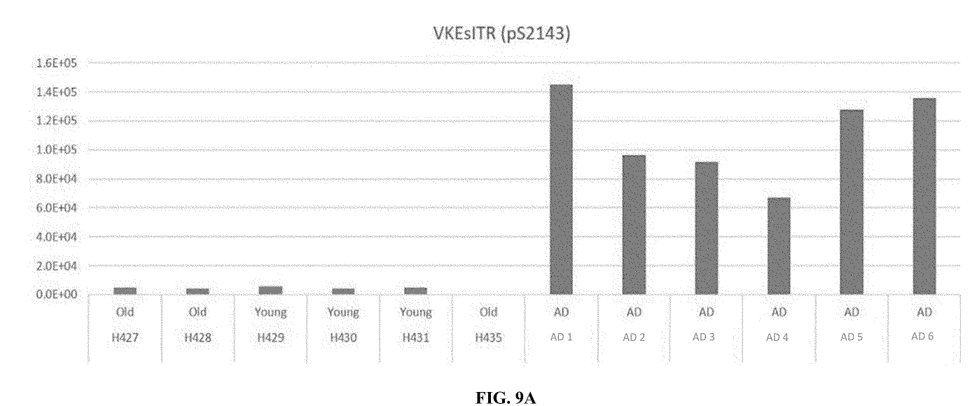

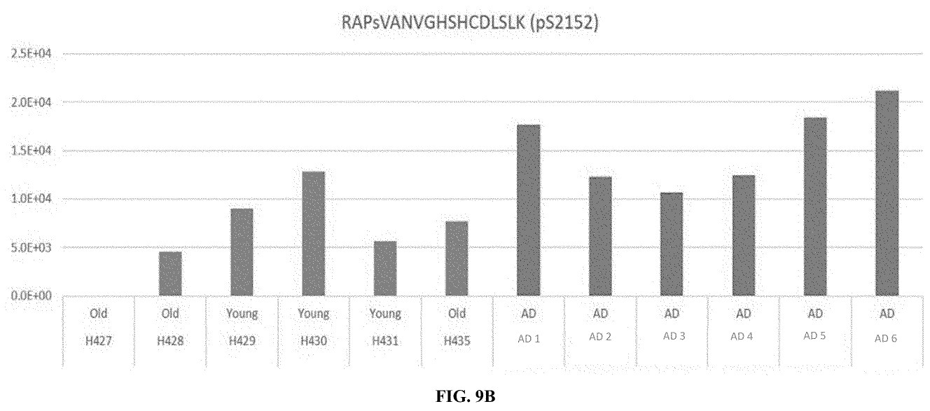

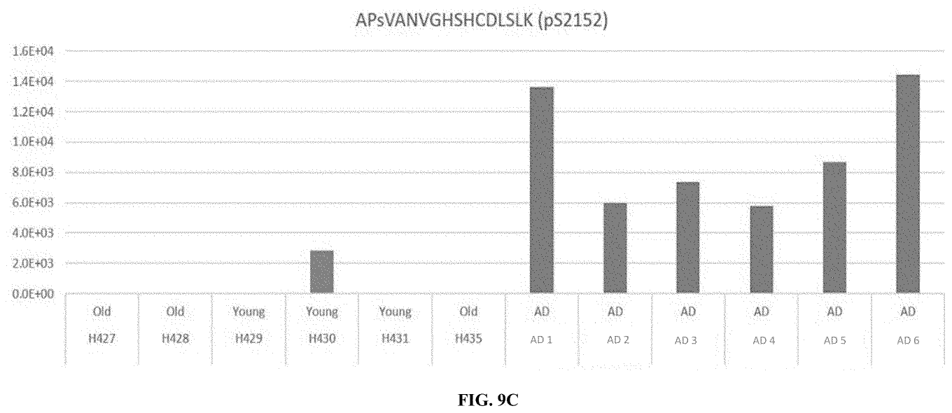

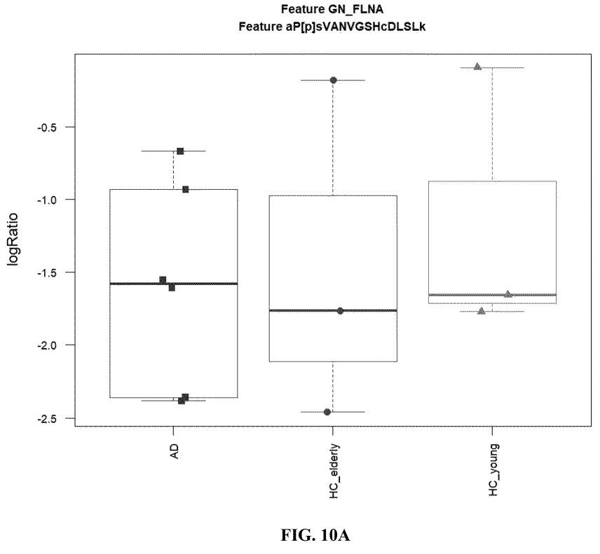

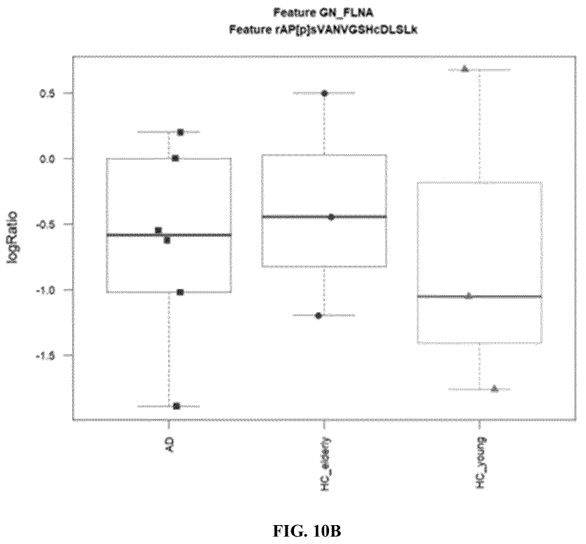

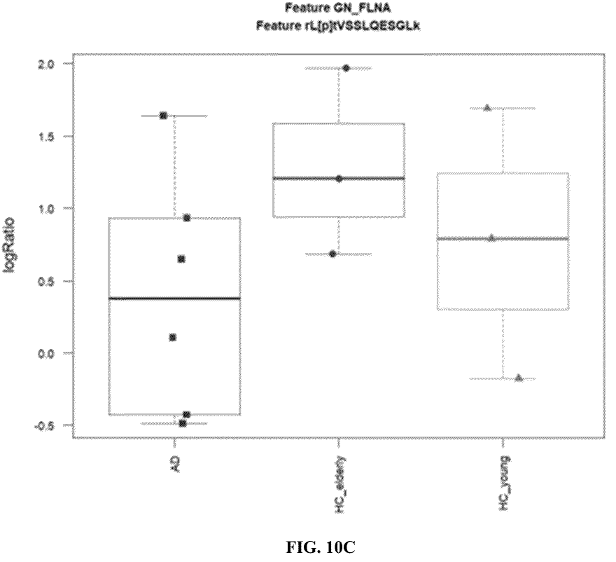

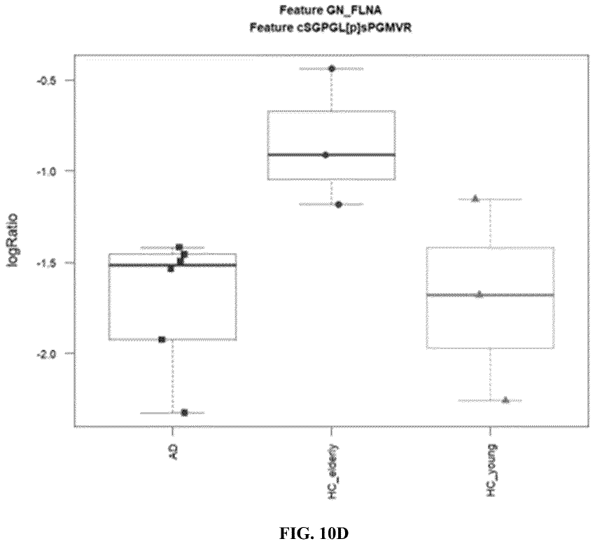

Filamin A (FLNA), was successfully detected at the protein and the phosphopeptide level and was found to be significantly regulated in the comparison of AD patients with both elderly and young controls. The protein itself was quantified in standard searching for tryptic peptides by 85 PSMs from 46 peptides (Table 6) excluding the phosphopeptides. Of the 5 detected phosphopeptides, 4 could be quantified across all individuals (see, e.g., Table 7). The phosphopeptide aP[p]sVANVGSHcDLSLk did not deliver a sufficient number of data points for the control samples as signals of these were below the detectable range. However, when plotting reporter ion intensity values of the respective peptide spectral match it becomes evident that this peptide is nicely detected in the AD samples as opposed to the controls, consistent with the data generated for the peptide RAP[p]sVANVGSHcDLSLk that is covering the same phosphorylation site in the protein (S2152) (FIG. 9B).

TABLE 6

Quantified peptides (non-phosphorylated) of FLNA

| Protein Description | Number PSMs | Number Peptides | Contrast #1 | | Contrast #2 | | Contrast #3 | |
|---|---|---|---|---|---|---|---|---|
| | | | logFC | adj. p-value | logFC | adj. p-value | logFC | adj. p-value |
| Filamin A | 85 | 46 | 1.78 | 6.95E-07 | 1.68 | 9.89E-07 | 1.73 | 1.03E-07 |

TABLE 7

Phosphopeptides of Target Protein FLNA

| Amino acid sequence | Phosphosite | Contrast #1 | | Contrast #2 | | Contrast #3 | |
|---|---|---|---|---|---|---|---|
| | | logFC | adj. p-value | logFC | adj. p-value | logFC | adj. p-value |
| vkE[p]sITR | pS2143 | 6.38 | 1.80E-05 | 5.47 | 2.45E-05 | 5.92 | 2.14E-06 |
| aP[p]sVANVGSHcDLSLk | pS2152 | Not quantified, as too many missing values in control samples. | | | | | |
| cSGPGL[p]sPGMVR | pS1459 | 0.21 | 5.30E-01 | 0.96 | 3.25E-03 | 0.59 | 1.77E-02 |
| iPEISIQDMTAQV[?p]t[?p]PSGK | pT2179 or pS2180 | 1.52 | 8.51E-02 | 1.71 | 5.28E-02 | 1.62 | 2.21E-02 |
| rAP[p]sVANVGSHcDLSLk | pS2152 | 0.57 | 4.47E-01 | -0.17 | 8.54E-01 | 0.20 | 7.52E-01 |

Overview of phosphopeptides with determined phosphosites of the target protein FLNA. Site positions that are ambiguously localized, with a confidence score below the set threshold (75%), are indicated by the “?” character. Whereas the strong regulation of FLNA peptides is encouraging, there is a risk that signals arise due to post-collection artifacts e.g. platelet activation since AD and control plasma samples were prepared differently. To explore this, and provide an expanded panel of biomarkers to control for artefactual signals, we performed further experiments as set out in Examples 2 and 3.

### Example 2: Analysis of Changes in FLNA, Phosphorylated FLNA and Other Proteins in Histopaque® 1077 Plasma Samples

In this example we used fresh plasma samples from AD and control cohorts prepared by an identical process expected to trigger platelet activation. Again, 6 samples from controls (3 elderly controls, 3 young cognitive intact) (FLNA band negative or ‘-’) and 6 AD patients (FLNA band positive or ‘+’)) were analysed using TMTcalibrator™ with the same AD brain trigger as described in Example 1. Whole blood from AD and control cohorts was collected and processed using the Histopaque® 1077 process described previously.

### Sample Processing

After thawing of plasma samples, all processing steps were performed as described in Example 1. Mass spectrometry was performed using an inclusion list for all FLNA peptides detected in Example 1.

### Results

Using a dedicated Filamin A inclusion scheme during MS acquisition, we obtained good coverage of the Filamin A sequence (approx. 40%). No significant changes in abundance could be observed for the protein as a whole, although particular parts of the sequence showed varying changes in abundance across the different groups. We also only observed subtle differences in expression levels of two of the quantified phosphosites.

In addition to FLNA, we quantified a total of 29,786 peptides and 3,297 phosphosites derived from 3,493 proteins, providing a useful resource for the identification of Alzheimer’s disease pathology dependent plasma markers. The statistical analysis indicated the existence of panels of phosphopeptide and protein features driving the separation of the experimental classes and these represent candidate biomarkers independent of platelet activation status. The functional enrichment indicated changes in the cytoskeletal organization, complement cascade regulation and lipoprotein metabolism.

### FLNA Coverage

FLNA was identified with 220 PSMs for 77 (phospho)peptides and quantified in the standard search for tryptic and semi-tryptic peptides by 158 PSMs from 54 peptides (Table 4) excluding the phosphopeptides. All of the four detected phosphopeptides could be quantified across all individuals (Table 5 contains statistical data). Two peptides were only identified in control samples: AGQSAAGAAPGGGVDTR (aa 8 - 24, SEQ ID NO: xx) was identified in 4 out of 6 controls, 3 young and 1 elderly; and DNGNGTYSCSYVPR (SEQ ID NO: vv) was identified in 1 young control only. One peptide, AVPTGDASK (aa 1636 - 1644; SEQ ID NO:CC), was exclusively identified in Alzheimer’s disease samples, with values obtained for 5 out of 6 patients.

Surprisingly, when the level of platelet activation was likely to be similar in AD and control samples due to the use of Histopaque® 1077 preparations, the elevation previously seen in AD was lost. In fact, the AD samples had lower levels of free plasma FLNA suggesting AD platelets were less prone to activation than platelets from cognitively healthy controls. We believe this loss of regulation is due to significant release of FLNA in the control plasma samples in this study.

TABLE 8

Quantification of FLNA in Histopaque® 1077 plasma samples

| Protein Descriptions | Number PSMs | Number Peptides | AD vs Old | | | AD vs Young | | | AD vs All Control | | |
|---|---|---|---|---|---|---|---|---|---|---|---|
| | | | logFC | p-value | adj. p-value | logFC | p-value | adj. p-value | logFC | p-value | adj. p-value |
| Filamin A | 158 | 54 | -0.26 | 5.08E-01 | 8.49E-01 | -0.09 | 8.11E-01 | 9.50E-01 | -0.18 | 5.77E-01 | 8.69E-01 |

We also identified four phosphorylated FLNA peptides, three of which were seen in Example 1 and one which was unique to the current analysis. Unlike the previous study, levels of the two peptides containing pS2152 did not show a regulation between the AD and control groups, although there was a trend for higher expression of the “RAPSVAN” peptide in AD and elderly controls suggesting this can be more related to disease than sample processing, at least in the periphery. Conversely, the peptide “CSGPG” containing pS1459 showed a strong up-regulation in healthy elderly that can be indicative of a protective post-translational modification. Interestingly, this was also seen in the previous study, although there was also an elevation in the AD group which was not seen this time and can instead suggest this site is produced artefactually, requiring further evaluation. The threonine phosphosite at residue T2336 showed a similar behaviour as the aforementioned serine phosphorylation: the levels detected in the elderly control were higher compared to both the Alzheimer’s disease patients, as well as the young healthy controls.

TABLE 9

Quantified phosphorylated peptides from FLNA in Histopaque® 1077 plasma

| Protein | ptmRS | Global Position | Safe Site | Squat Sequence | PSMs | AD vs CTRL | | |
|---|---|---|---|---|---|---|---|---|
| | | | | | | logFC | p-value | adj. p-value |
| Filamin A | S3(Phospho): 100 | P21333:S2152 | S3 | AP[p]SVANVGSHCDLSLK | 1 | -0.26 | 6.32E-01 | 8.86E-01 |
| Filamin A | S7(Phospho): 100 | P21333:S1459 | S7 | CSGPGL[p]SPGMVR | 2 | -0.41 | 8.55E-02 | 5.07E-01 |
| Filamin A | S4(Phospho): 100 | P21333:S2152 | S4 | RAP[p]SVANVGSHCDLSLK | 1 | -0.10 | 8.58E-01 | 9.64E-01 |
| Filamin A | T3(Phospho): 100 | P21333:T2336 | T3 | RL[p]TVSSLQESGLK | 1 | -0.63 | 2.22E-01 | 6.64E-01 |

### Example 3: TMTcalibrator™ Analysis of AD and Control EDTA Plasma Samples

The results of Example 2 provided evidence that some regulation of protein expression can be driven by the sample preparation method. We therefore completed our study by analysing fresh samples of AD and control plasma samples that had all been prepared from EDTA blood collection tubes under identical conditions. All other conditions were as described in Examples 1 and 2. We expected the level of platelet activation to be the lowest in this set of samples, which better reflect standard clinical practice.

### Results

FLNA was successfully detected at the protein and phosphopeptide level. We detected 60 unmodified peptides associated with FLNA, of which 54 were unique to FLNA and 42 could be quantified in all 12 samples. The protein was found to be significantly upregulated (about2-fold) in the AD samples compared to the healthy control groups. Three of four detected phosphopeptides associated with FLNA were used for quantification. These peptides carry modifications at the phosphorylation sites pS1459, pS2143 and pS2152. All three phosphopeptide features were more abundant in the AD groups, as was seen in Example 1, but the extent of regulation was not as strong here. We hypothesize that this reflects the true biological difference in FLNA abundance in patients with Alzheimer’s disease. Where platelet activation is significant, the amount of released FLNA is sufficient to mimic that seen in disease and even reverse the relative abundance signature between healthy individuals and AD patients.

TABLE 10

Relative expression of phosphorylated FLNA peptides in EDTA plasma

| Protein | ptmRS | Global Position | Safe Site | Squat Sequence | PSMs | AD vs CTL | | |
|---|---|---|---|---|---|---|---|---|
| | | | | | | logFC | p-value | adj. p-value |
| Filamin A | S4(Phospho): 100 | P21333:S2143 \|O7 5369.S2098 | S4 | vkE[p]sITR | 2 | 2.76 | 4.32E-04 | 2.79E-02 |
| Filamin A | S3(Phospho): 100 | P21333:S2152 | S3 | aP[p]sVANVGSHcDLSLk | 2 | 0.87 | 4.84E-03 | 1.02E-01 |
| Filamin A | S7(Phospho): 100 | P21333:S1459 | S7 | cSGPGL[p]sPGMVR | 3 | 1.42 | 1.22E-02 | 1.57E-01 |

TABLE 11

Relative expression of non-phosphorylated FLNA in EDTA plasma

| Protein | #PSMs | #Peptides | AD vs Old | | | AD vs Young | | | AD vs All Control | | |
|---|---|---|---|---|---|---|---|---|---|---|---|
| | | | logFC | p-value | adj. p-value | logFC | p-value | adj. p-value | logFC | p-value | adj. p-value |
| Filamin A | 116 | 42 | 0.96 | 2.19E-08 | 3.81E-05 | 1.01 | 1.29E-08 | 4.4810-05 | 0.98 | 4.55E-09 | 7.92E-06 |

In addition to FLNA, we quantified a total of 31,382 peptides, 7,677 phosphopeptides and 3,478 proteins. The statistical analysis revealed multiple significantly regulated phosphopeptide and protein features driving the separation of the experimental classes. The functional enrichment indicated changes in the platelet activation, cell-extracellular matrix interaction and cell junction organization.

We obtained good characterization of Filamin A in AD and control K2EDTA plasma. Unlike the results obtained with plasma prepared with the Histopaque® 1077 method, quantitative differences in Filamin A abundance and multiple other protein features were detected in the current study. The results were comparable to the first study (40-224), albeit the observed differences were less pronounced.

We found various FLNA-associated tryptic peptides with good regulation between the AD and control groups. We believe that these results can serve as good basis to develop a targeted mass spectrometry method for FLNA with diagnostic and prognostic utility in the context of selection and monitoring of patients for treatment with simufilam.

### Example 4: Comparison of Protein Expressions in AD and Cognitively Normal Individuals Across Three Different Sample Preparation Methods

We compared the relative expression of different proteins, including FLNA and phosphorylated peptides of FLNA in the different plasma groups of AD Histopaque® 1077 vs Control EDTA, AD Histopaque® 1077 vs Control Histopaque® 1077, and AD EDTA vs Control EDTA. This confirmed the suitability of FLNA, and in particular phosphorylated eptiopes of FLNA to serve as peripheral biomarkers of Alzheimer’s disease. However, it is evident that the potential activation of platelets using non-standard plasma preparation can potentially mimic the elevated FLNA levels seen in AD and we therefore searched for peptides and proteins whose levels were elevated in Histopaque® 1077 plasma but were not detected in EDTA plasma. We also focused on a specific subset of such proteins with documented associations with platelet biology.

The relative expression of FLNA is clearly affected by the use of Histopaque® 1077 for plasma preparation. In general, we see that the relative level of phosphorylated FLNA is particularly increased in cognitively healthy individuals compared to AD patients (Table 12). In Example 1 Histopaque® 1077 was used to prepare AD plasma whereas controls were prepared using EDTA. All three of the phosphorylated FLNA peptides detected in Example 1 were found increased in the AD group, but two of these showed a reduced level in AD vs controls when both were prepared by Histopaque® 1077. For pS1459 of FLNA the extent of regulation in AD vs control groups was seen to be further extended in EDTA-prepared plasma samples tested in Example 3.

TABLE 12

Relative expression of FLNA phosphopeptides in plasma from AD patients compared to controls from Examples 1 - 3

| | | Log2 Fold-change AD vs CTL | | |
|---|---|---|---|---|
| Peptide Sequence | Phosphorylated Residue | Example 1 | Example 2 | Example 3 |
| cSGPGL[p]sPGMVR | S1459 | 0.586 | -0.407 | 1.421 |
| vkE[p]sITR | S2143 | N D | ND | 2.758 |
| aP [p] sVANVGSHcDLSLk | S2152 | ND | -0.263 | 0.874 |
| rAP[p]sVANVGSHcDLSLk | S2152 | 0.2.02 | -0.100 | ND |
| iPEISIQDMTAQV[?p] t [?p] sPSGk | T2179?; S2180? | 1.619 | ND | ND |
| rL [p] tVSSLQESGLk | T2336 | ND | -0.625 | ND |

We also identified three peptides that could be used to assess the level of artefactual platelet activation during preparation of plasma or serum. These peptides were derived from proteins with annotations to roles in platelet biology, and were differentially expressed in AD vs control groups when Histopaque® 1077 was used in the AD samples only, were not regulated when Histopaque® 1077 was used for both cohorts, and were not detected in either group when EDTA was used to prepare plasma. In this respect, the inclusion of these peptides or their respective proteins in methods of the present invention can be used to indicate samples where platelet activation has occurred and a fresh sample should be tested where the absence of these peptides/proteins is indicative of a good sample.

TABLE 13

Peptides indicating excessive platelet activation

| | | Log2 Fold-change AD vs Control | | |
|---|---|---|---|---|
| Peptide Sequence | Protein | Example 1 | Example 2 | Example 3 |
| aEYSPcR | Integrin alpha-IIb | 4.41 | -0.28 | ND |
| gALHDENTcNR | Integrin beta-3 | 5.33 | 0.00 | ND |
| eYVNV [p] sQEL HPGAAk | Linker for activation of T-cells family member 1 | 3.35 | -1.08 | ND |

Throughout this specification, various patents, patent applications and/or other types of publications (e.g., journal articles and books) are referenced. The disclosure of all patents, patent applications, and publications cited herein are hereby incorporated by reference in their entirety for all purposes.

1. A biomarker panel comprising one or more phosphorylated peptides obtained from in vitro digestion of a Filamin A protein having the polypeptide sequence of SEQ ID NO: 1.

2. The biomarker panel of claim 1, wherein the Filamin A is contained within or obtained from a biological fluid or tissue sample taken from a subject suspected of having a neurological disease.

3. The biomarker panel of claim 3, wherein the neurological disease is Alzheimer’s disease.

4. The biomarker panel of claim 1, wherein the phosphorylated peptide is phosphorylated at a residue corresponding to serine 2152 of SEQ ID NO: 1.

5. The biomarker panel of claim 1, wherein the phosphorylated peptide has the amino acid sequence of SEQ ID NO: 2, SEQ ID NO: 3, SEQ ID NO: 4, or SEQ ID NO: 5.

6. The biomarker panel of claim 1, wherein the biomarker panel comprises a plurality of phosphorylated fragments of FLNA, optionally two or more fragments or three or more fragments.

7. The biomarker panel of claim 1, wherein the biomarker panel comprises at least 2, 3, 4, 5, 6, 7, 8, 9, or 10 different phosphorylated fragments of FLNA.

8. The biomarker panel of claim 1, wherein the biomarker panel comprises two or more phosphorylated peptides, each comprising a fragment of FLNA and having the amino acid sequence of any one of SEQ ID NOs: 2-5, wherein at least one of the two or more phosphorylated peptides is phosphorylated at a position corresponding to serine 2152 of SEQ ID NO: 1.

9. The biomarker panel of claim 1, wherein the phosphorylated peptide is phosphorylated at a position corresponding to serine 2143 of SEQ ID NO: 1.

10. The biomarker panel of claim 1, wherein the phosphorylated peptide has the amino acid sequence of SEQ ID NO: 6, SEQ ID NO: 7, SEQ ID NO: 8, or SEQ ID NO:9.

11. The biomarker panel of claim 1, wherein the biomarker panel comprises two or more phosphorylated peptides, each comprising a fragment of FLNA and having the amino acid sequence of any one of SEQ ID NOs: 6-9, wherein at least one of the two or more phosphorylated peptides is phosphorylated at a position corresponding to serine 2143 of SEQ ID NO: 1.

12. The biomarker panel of claim 1, wherein the phosphorylated peptide is phosphorylated at a position corresponding to serine 2180 of SEQ ID NO: 1.

13. The biomarker panel of claim 1, wherein the phosphorylated peptide has the amino acid sequence of SEQ ID NO: 10, SEQ ID NO: 11, or SEQ ID NO: 12.

14. The biomarker panel of claim 1, wherein the biomarker panel comprises two or more phosphorylated peptides, each comprising a fragment of FLNA and having the amino acid sequence of any one of SEQ ID NOs: 10-12, wherein at least one of the two or more phosphorylated peptides is phosphorylated at a position corresponding to serine 2180 of SEQ ID NO: 1.

15. A biomarker panel, wherein the biomarker panel comprises a plurality of phosphorylated peptides, wherein each phosphorylated peptide is a fragment of SEQ ID NO:1 and comprises a phosphorylation site at a position corresponding to serine 2143, 2152, and/or 2180 of SEQ ID NO: 1.

16. A biomarker panel, comprising a plurality of phosphorylated peptides, each having the sequence of SEQ ID Nos: 2-12 and being phosphorylated at a position corresponding to serine 2143, 2152, and/or 2180 of SEQ ID NO: 1.

17. A biomarker panel, comprising at least 2, 3, 4, 5, 6, 7, 8, 9, or 10 different phosphorylated peptides, each being phosphorylated at a site corresponding to serine residue 2143, 2152 and/or 2180 of SEQ ID NO: 1.

18. A biomarker panel, comprising: (i) one or more peptides obtained from in vitro digestion of Filamin A using a protease or combination of proteases, wherein each of the one or more peptides comprises an amino acid sequence present in SEQ ID NO:1; and (ii) one or more peptides obtained from in vitro digestion of one or more, optionally two or more of the proteins listed in Tables 1-4.

19. A method for the diagnosis/prognosis of a neurological disorder, comprising:
obtaining a bodily fluid or tissue sample from a subject;
digesting one or more proteins in the bodily fluid or tissue sample with one or more proteases; and
detecting and/or measuring the level of one or more peptides produced from the digestion, using mass spectrometry, wherein the one or more peptides are obtained from in vitro digestion of one or more of the proteins listed in Tables 1-4.

20. A method for monitoring the progression of a neurological disorder, comprising:
(a) obtaining a bodily fluid or tissue sample from a subject at a first time point;
(b) digesting one or more proteins in the bodily fluid or tissue sample with one or more proteases;
(c) detecting and/or measuring the level of one or more peptides produced from the digestion, using mass spectrometry, wherein the one or more peptides are obtained from in vitro digestion of one or more of the proteins listed in Tables 1-4; and
(d) repeating steps (a)-(c) at a second time point.


SEQUENCE LISTING (186 sequences)

SEQ ID NO: 1 - protein, 2647 aa, Homo sapiens
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTVSIGGHGLGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 2 - protein, 16 aa, Homo sapiens
  MOD_RES 3: note = PHOSPHORYLATION
APSVANVGSHCDLSLK

SEQ ID NO: 3 - protein, 17 aa, Homo sapiens
  MOD_RES 4: note = PHOSPHORYLATION
RAPSVANVGSHCDLSLK

SEQ ID NO: 4 - protein, 26 aa, Homo sapiens
  MOD_RES 10: note = PHOSPHORYLATION
SITRRRRAPSVANVGSHCDLSLKIPE

SEQ ID NO: 5 - protein, 38 aa, Homo sapiens
  MOD_RES 36: note = PHOSPHORYLATION
NIKFADQHVPGSPFSVKVTGEGRVKESITRRRRAPSVA

SEQ ID NO: 6 - protein, 7 aa, Homo sapiens
  MOD_RES 4: note = PHOSPHORYLATION
VKESITR

SEQ ID NO: 7 - protein, 5 aa, Homo sapiens
  MOD_RES 2: note = PHOSPHORYLATION
ESITR

SEQ ID NO: 8 - protein, 26 aa, Homo sapiens
  MOD_RES 1: note = PHOSPHORYLATION
SITRRRRAPSVANVGSHCDLSLKIPE

SEQ ID NO: 9 - protein, 38 aa, Homo sapiens; identical to SEQ ID NO: 5
  MOD_RES 27: note = PHOSPHORYLATION

SEQ ID NO: 10 - protein, 19 aa, Homo sapiens
  MOD_RES 15: note = PHOSPHORYLATION
IPEISIQDMTAQVTSPSGK

SEQ ID NO: 11 - protein, 19 aa, Homo sapiens
  MOD_RES 12: note = PHOSPHORYLATION
ISIQDMTAQVTSPSGKTHE

SEQ ID NO: 12 - protein, 41 aa, Homo sapiens
  MOD_RES 27: note = PHOSPHORYLATION
NTVGSHCDLSLKIPEISIQDMTAQVTSPSGKTHEAEIVEGE

SEQ ID NO: 13 - protein, 12 aa, Homo sapiens
  MOD_RES 7: note = PHOSPHORYLATION
CSGPGLSPGMVR

SEQ ID NO: 14 - protein, 77 aa, Homo sapiens
  MOD_RES 41: note = PHOSPHORYLATION
AGTYSLNVTYGGHQVPGSPFKVPVHDVTDASKVKCSGPGLSPGMVRANLPQSFQVDTSKAGVAPLQVKVQGPKGLVE

SEQ ID NO: 15 - protein, 41 aa, Homo sapiens
  MOD_RES 35: note = PHOSPHORYLATION
NVTYGGHQVPGSPFKVPVHDVTDASKVKCSGPGLSPGMVRA

SEQ ID NO: 16 - protein, 372 aa, Homo sapiens
MAYHSFLVEPISCHAWNKDRTQIAICPNNHEVHIYEKSGAKWTKVHELKEHNGQVTGIDWAPESNRIVTCGTDRNAYVWTLKGRTWKPTLVILRINRAARCVRWAPNENKFAVGSGSRVISICYFEQENDWWVCKHIKKPIRSTVLSLDWHPNNVLLAAGSCDFKCRIFSAYIKEVEERPAPTPWGSKMPFGELMFESSSSCGWVHGVCFSASGSRVAWVSHDSTVCLADADKKMAVATLASETLPLLALTFITDNSLVAAGHDCFPVLFTYDAAAGMLSFGGRLDVPKQSSQRGLTARERFQNLDKKASSEGGTAAGAGLDSLHKNSVSQISVLSGGKAKCSQFCTTGMDGGMSIWDVKSLESALKDLKIK

SEQ ID NO: 17 - protein, 151 aa, Homo sapiens
MSKNTVSSARFRKVDVDEYDENKFVDEEDGGDGQAGPDEGEVDSCLRQGNMTAALQAALKNPPINTKSQAVKDRAGSIVLKVLISFKANDIEKAVQSLDKNGVDLLMKYIYKGFESPSDNSSAMLLQWHEKALAAGGVGSIVRVLTARKTV

SEQ ID NO: 18 - protein, 362 aa, Homo sapiens
MAAAEEEPKPKKLKVEAPQALRENILFGMGNPLLDISAVVDKDFLDKYSLKPNDQILAEDKHKELFDELVKKFKVEYHAGGSTQNSIKVAQWMIQQPHKAATFFGCIGIDKFGEILKRKAAEAHVDAHYYEQNEQPTGTCAACITGDNRSLIANLAAANCYKKEKHLDLEKNWMLVEKARVCYIAGFFLTVSPESVLKVAHHASENNRIFTLNLSAPFISQFYKESLMKVMPYVDILFGNETEAATFAREQGFETKDIKEIAKKTQALPKMNSKRQRIVIFTQGRDDTIMATESEVTAFAVLDQDQKEIIDTNGAGDAFVGGFLSQLVSDKPLTECIRAGHYAASIIIRRTGCTFPEKPDFH

SEQ ID NO: 19 - protein, 323 aa, Homo sapiens
MASIWVGHRGTVRDYPDFSPSVDAEAIQKAIRGIGTDEKMLISILTERSNAQRQLIVKEYQAAYGKELKDDLKGDLSGHFEHLMVALVTPPAVFDAKQLKKSMKGAGTNEDALIEILTTRTSRQMKDISQAYYTVYKKSLGDDISSETSGDFRKALLTLADGRRDESLKVDEHLAKQDAQILYKAGENRWGTDEDKFTEILCLRSFPQLKLTFDEYRNISQKDIVDSIKGELSGHFEDLLLAIVNCVRNTPAFLAERLHRALKGIGTDEFTLNRIMVSRSEIDLLDIRTEFKKHYGYSLYSAIKSDTSGDYEITLLKICGGDD

SEQ ID NO: 20 - protein, 565 aa, Homo sapiens
MAEGKAGGAAGLFAKQVQKKFSRAQEKVLQKLGKAVETKDERFEQSASNFYQQQAEGHKLYKDLKNFLSAVKVMHESSKRVSETLQEIYSSEWDGHEELKAIVWNNDLLWEDYEEKLADQAVRTMEIYVAQFSEIKERIAKRGRKLVDYDSARHHLEAVQNAKKKDEAKTAKAEEEFNKAQTVFEDLNQELLEELPILYNSRIGCYVTIFQNISNLRDVFYREMSKLNHNLYEVMSKLEKQHSNKVFVVKGLSSSSRRSLVISPPVRTATVSSPLTSPTSPSTLSLKSESESVSATEDLAPDAAQGEDNSEIKELLEEEEIEKEGSEASSSEEDEPLPACNGPAQAQPSPTTERAKSQEEVLPSSTTPSPGGALSPSGQPSSSATEVVLRTRTASEGSEQPKKRASIQRTSAPPSRPPPPRATASPRPSSGNIPSSPTASGGGSPTSPRASLGTGTASPRTSLEVSPNPEPPEKPVRTPEAKENENIHNQNPEELCTSPTLMTSQVASEPGEAKKMEDKEKDNKLISANSSEGQDQLQVSMVPENNNLTAPEPQEEVSTSENPQL

SEQ ID NO: 21 - protein, 793 aa, Homo sapiens
MDDFERRRELRRQKREEMRLEAERIAYQRNDDDEEEAARERRRRARQERLRQKQEEESLGQVTDQVEVNAQNSVPDEEAKTTTTNTQVEGDDEAAFLERLARREERRQKRLQEALERQKEFDPTITDASLSLPSRRMQNDTAENETTEKEEKSESRQERYEIEETETVTKSYQKNDWRDAEENKKEDKEKEEEEEEKPKRGSIGENQVEVMVEEKTTESQEETVVMSLKNGQISSEEPKQEEEREQGSDEISHHEKMEEEDKERAEAERARLEAEERERIKAEQDKKIADERARIEAEEKAAAQERERREAEERERMREEEKRAAEERQRIKEEEKRAAEERQRIKEEEKRAAEERQRIKEEEKRAAEERQRARAEEEEKAKVEEQKRNKQLEEKKHAMQETKIKGEKVEQKIEGKWVNEKKAQEDKLQTAVLKKQGEEKGTKVQAKREKLQEDKPTFKKEEIKDEKIKKDKEPKEEVKSFMDRKKGFTEVKSQNGEFMTHKLKHTENTFSRPGGRASVDTKEAEGAPQVEAGKRLEELRRRRGETESEEFEKLKQKQQEAALELEELKKKREERRKVLEEEEQRRKQEEADRKLREEEEKRRLKEEIERRRAEAAEKRQKMPEDGLSDDKKPFKCFTPKGSSLKIEERAEFLNKSVQKSSGVKSTHQAAIVSKIDSRLEQYTSAIEGTKSAKPTKPAASDLPVPAEGVRNIKSMWEKGNVFSSPTAAGTPNKETAGLKVGVSSRINEWLTKTPDGNKSPAPKPSDLRPGDVSSKRNLWEKQSVDKVTSPTKV

SEQ ID NO: 22 - protein, 149 aa, Homo sapiens [sequence omitted]

SEQ ID NO: 23 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 24 - protein, 425 aa, Homo sapiens
MGEDAAQAEKFQHPGSDMRQEKPSSPSPMPSSTPSPSLNLGNTEEAIRDNSQVNAVTVLTLLDKLVNMLDAVQENQHKMEQRQISLEGSVKGIQNDLTKLSKYQASTSNTVSKLLEKSRKVSAHTRAVKERMDRQCAQVKRLENNHAQLLRRNHFKVLIFQEENEIPASVFVKQPVSGAVEGKEELPDENKSLEETLHTVDLSSDDDLPHDEEALEDSAEEKVEESRAEKIKRSSLKKVDSLKKAFSRQNIEKKMNKLGTKIVSVERREKIKKSLTSNHQKISSGKSSPFKVSPLTFGRKKVREGESHAENETKSEDLPSSEQMPNDQEEESFAEGHSEASLASALVEGEIAEEAAEKATSRGSNSGMDSNIDLTIVEDEEEESVALEQAQKVRYEGSYALTSEEAERSDGDPVQPAVLQVHQTS

SEQ ID NO: 25 - protein, 241 aa, Homo sapiens
MAEEQPQVELFVKAGSDGAKIGNCPFSQRLFMVLWLKGVTFNVTTVDTKRRTETVQKLCPGGQLPFLLYGTEVHTDTNKIEEFLEAVLCPPRYPKLAALNPESNTAGLDIFAKFSAYIKNSNPALNDNLEKGLLKALKVLDNYLTSPLPEEVDETSAEDEGVSQRKFLDGNELTLADCNLLPKLHIVQVVCKKYRGFTIPEAFRGVHRYLSNAYAREEFASTCPDDEEIELAYEQVAKALK

SEQ ID NO: 26 - protein, 613 aa, Homo sapiens
MCCWPLLLLWGLLPGTAAGGSGRTYPHRTLLDSEGKYWLGWSQRGSQIAFRLQVRTAGYVGFGFSPTGAMASADIVVGGVAHGRPYLQDYFTNANRELKKDAQQDYHLEYAMENSTHTIIEFTRELHTCDINDKSITDSTVRVIWAYHHEDAGEAGPKYHDSNRGTKSLRLLNPEKTSVLSTALPYFDLVNQDVPIPNKDTTYWCQMFKIPVFQEKHHVIKVEPVIQRGHESLVHHILLYQCSNNFNDSVLESGHECYHPNMPDAFLTCETVIFAWAIGGEGFSYPPHVGLSLGTPLDPHYVLLEVHYDNPTYEEGLIDNSGLRLFYTMDIRKYDAGVIEAGLWVSLFHTIPPGMPEFQSEGHCTLECLEEALEAEKPSGIHVFAVLLHAHLAGRGIRLRHFRKGKEMKLLAYDDDFDFNFQEFQYLKEEQTILPGDNLITECRYNTKDRAEMTWGGLSTRSEMCLSYLLYYPRINLTRCASIPDIMEQLQFIGVKEIYRPVTTWPFIIKSPKQYKNLSFMDAMNKFKWTKKEGLSFNKLVLSLPVNVRCSKTDNAEWSIQGMTALPPDIERPYKAEPLVCGTSSSSSLHRDFSINLLVCLLLLSCTLSTKSL

SEQ ID NO: 27 - protein, 102 aa, Homo sapiens
MSSPPEGKLETKAGHPPAVKAGGMRIVQKHPHTGDTKEEKDKDDQEWESPSPPKPTVFISGVIARGDKDFPPAAAQVAHQKPHASMDKHPSPRTQHIQQPRK

SEQ ID NO: 28 - protein, 348 aa, Homo sapiens
MNMTQARVLVAAVVGLVAVLLYASIHKIEEGHLAVYYRGGALLTSPSGPGYHIMLPFITTFRSVQTTLQTDEVKNVPCGTSGGVMIYIDRIEVVNMLAPYAVFDIVRNYTADYDKTLIFNKIHHELNQFCSAHTLQEVYIELFDQIDENLKQALQKDLNLMAPGLTIQAVRVTKPKIPEAIRRNFELMEAEKTKLLIAAQKQKVVEKEAETERKKAVIEAEKIAQVAKIRFQQKVMEKETEKRISEIEDAAFLAREKAKADAEYYAAHKYATSNKHKLTPEYLELKKYQAIASNSKIYFGSNIPNMFVDSSCALKYSDIRTGRESSLPSKEALEPSGENVIQNKESTG

SEQ ID NO: 29 - protein, 885 aa, Homo sapiens
MGCAPSIHVSQSGVIYCRDSDESSSPRQTTSVSQGPAAPLPGLFVQTDAADAIPPSRASGPPSVARVRRARTELGSGSSAGSAAPAATTSRGRRRHCCSSAEAETQTCYTSVKQVSSAEVRIGPMRLTQDPIQVLLIFAKEDSQSDGFWWACDRAGYRCNIARTPESALECFLDKHHEIIVIDHRQTQNFDAEAVCRSIRATNPSEHTVILAVVSRVSDDHEEASVLPLLHAGFNRRFMENSSIIACYNELIQIEHGEVRSQFKLRACNSVFTALDHCHEAIEITSDDHVIQYVNPAFERMMGYHKGELLGKELADLPKSDKNRADLLDTINTCIKKGKEWQGVYYARRKSGDSIQQHVKITPVIGQGGKIRHFVSLKKLCCTTDNNKQIHKIHRDSGDNSQTEPHSFRYKNRRKESIDVKSISSRGSDAPSLQNRRYPSMARIHSMTIEAPITKVINIINAAQENSPVTVAEALDRVLEILRTTELYSPQLGTKDEDPHTSDLVGGLMTDGLRRLSGNEYVFTKNVHQSHSHLAMPITINDVPPCISQLLDNEESWDFNIFELEAITHKRPLVYLGLKVFSRFGVCEFLNCSETTLRAWFQVIEANYHSSNAYHNSTHAADVLHATAFFLGKERVKGSLDQLDEVAALIAATVHDVDHPGRTNSFLCNAGSELAVLYNDTAVLESHHTALAFQLTVKDTKCNIFKNIDRNHYRTLRQAIIDMVLATEMTKHFEHVNKFVNSINKPMAAEIEGSDCECNPAGKNFPENQILIKRMMIKCADVANPCRPLDLCIEWAGRISEEYFAQTDEEKRQGLPVVMPVFDRNTCSIPKSQISFIDYFITDMFDAWDAFAHLPALMQHLADNYKHWKTLDDLKCKSLRLPSDS

SEQ ID NO: 30 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 31 - protein, 1039 aa, Homo sapiens
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERAIPIWWVLVGVLGGLLLLTILVLAMWKVGFFKRNRPPLEEDDEEGE

SEQ ID NO: 32 - protein, 788 aa, Homo sapiens
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTANNPLYKEATSTFTNITYRGT

SEQ ID NO: 33 - protein, 721 aa, Homo sapiens
MGTPASGRKRTPVKDRFSAEDEALSNIAREAEARLAAKRAARAEARDIRMRELERQQKEYSLHSFDRKWGQIQKWLEDSERARYSHRSSHHRPYLGVEDALSIRSVGSHRYDMFKDRSSRLSSLNHSYSHSHGMKKRSSDSHKDLLSGLYFDQRNYSSLRHSKPTSAYYTRQSSSLYSDPLATYKSDRASPTANSGLLRSASLASLYNGGLYNPYGPRTPSECSYYSSRISSARSSPGFTNDDTASIVSSDRASRGRRESVVSAADYFSRSNRRGSVVSEVDDISIPDLSSLDEKSDKQYAENYTRPSSRNSASATTPLSGNSSRRGSGDTSSLIDPDTSLSELRDIYDLKDQIQDVEGRYMQGLKELKESLSEVEEKYKKAMVSNAQLDNEKNNLIYQVDTLKDVIEEQEEQMAEFYRENEEKSKELERQKHMCSVLQHKMEELKEGLRQRDELIEEKQRMQQKIDTMTKEVFDLQETLLWKDKKIGALEKQKEYIACLRNERDMLREELADLQETVKTGEKHGLVIIPDGTPNGDVSHEPVAGAITVVSQEAAQVLESAGEGPLDVRLRKLAGEKEELLSQIRKLKLQLEEERQKCSRNDGTVGDLAGLQNGSDLQFIEMQRDANRQISEYKFKLSKAEQDITTLEQSISRLEGQVLRYKTAAENAEKVEDELKAEKRKLQRELRTALDKIEEMEMTNSHLAKRLEKMKANRTALLAQQ

SEQ ID NO: 34 - protein, 379 aa, Homo sapiens
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFCMLMPEENFTADHPFLFFIRHNSSGSILFLGRFSSP

SEQ ID NO: 35 - protein, 480 aa, Homo sapiens
MIRAAPPPLFLLLLLLLLLVSWASRGEAAPDQDEIQRLPGLAKQPSFRQYSGYLKGSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 36 - protein, 182 aa, Homo sapiens
MRRMWATQGLAVALALSVLPGSRALRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASARTDL

SEQ ID NO: 37 - protein, 577 aa, Homo sapiens
MPKTISVRVTTMDAELEFAIQPNTTGKQLFDQVVKTIGLREVWFFGLQYQDTKGFSTWLKLNKKVTAQDVRKESPLLFKFRAKFYPEDVSEELIQDITQRLFFLQVKEGILNDDIYCPPETAVLLASYAVQSKYGDFNKEVHKSGYLAGDKLLPQRVLEQHKLNKDQWEERIQVWHEEHRGMLREDAVLEYLKIAQDLEMYGVNYFSIKNKKGSELWLGVDALGLNIYEQNDRLTPKIGFPWSEIRNISFNDKKFVIKPIDKKAPDFVFYAPRLRINKRILALCMGNHELYMRRRKPDTIEVQQMKAQAREEKHQKQMERAMLENEKKKREMAEKEKEKIEREKEELMERLKQIEEQTKKAQQELEEQTRRALELEQERKRAQSEAEKLAKERQEAEEAKEALLQASRDQKKTQEQLALEMAELTARISQLEMARQKKESEAVEWQQKAQMVQEDLEKTRAELKTAMSTPHVAEPAENEQDEQDENGAEASADLRADAMAKDRSEEERTTEAEKNERVQKHLKALTSELANARDESKKTANDMIHAENMRLGRDKYKTLRQIRQGNTKQRIDEFESM

SEQ ID NO: 38 - protein, 151 aa, Homo sapiens
MCDFTEDQTAEFKEAFQLFDRTGDGKILYSQCGDVMRALGQNPTNAEVLKVLGNPKSDEMNVKVLDFEHFLPMLQTVAKNKDQGTYEDYVEGLRVFDKEGNGTVMGAEIRHVLVTLGEKMTEEEVEMLVAGHEDSNGCINYEAFVRHILSG

SEQ ID NO: 39 - protein, 118 aa, Homo sapiens
MCDKEFMWALKNGDLDEVKDYVAKGEDVNRTLEGGRKPLHYAADCGQLEILEFLLLKGADINAPDKHHITPLLSAVYEGHVSCVKLLLSKGADKTVKGPDGLTAFEATDNQAIKALLQ

SEQ ID NO: 40 - protein, 329 aa, Homo sapiens
MTTQQIDLQGPGPWGFRLVGGKDFEQPLAISRVTPGSKAALANLCIGDVITAIDGENTSNMTHLEAQNRIKGCTDNLTLTVARSEHKVWSPLVTEEGKRHPYKMNLASEPQEVLHIGSAHNRSAMPFTASPASSTTARVITNQYNNPAGLYSSENISNFNNALESKTAASGVEANSRPLDHAQPPSSLVIDKESEVYKMLQEKQELNEPPKQSTSFLVLQEILESEEKGDPNKPSGFRSVKAPVTKVAASIGNAQKLPMCDKCGTGIVGVFVKLRDRHRHPECYVCTDCGTNLKQKGHFFVEDQIYCEKHARERVTPPEGYEVVTVFPK

SEQ ID NO: 41 - protein, 1756 aa, Homo sapiens
MNSLFRKRNKGKYSPTVQTRSISNKELSELIEQLQKNADQVEKNIVDTEAKMQSDLARLQEGRQPEHRDVTLQKVLDSEKLLYVLEADAAIAKHMKHPQGDMIAEDIRQLKERVTNLRGKHKQIYRLAVKEVDPQVNWAALVEEKLDKLNNQSFGTDLPLVDHQVEEHNIFHNEVKAIGPHLAKDGDKEQNSELRAKYQKLLAASQARQQHLSSLQDYMQRCTNELYWLDQQAKGRMQYDWSDRNLDYPSRRRQYENFINRNLEAKEERINKLHSEGDQLLAAEHPGRNSIEAHMEAVHADWKEYLNLLICEESHLKYMEDYHQFHEDVKDAQELLRKVDSDLNQKYGPDFKDRYQIELLLRELDDQEKVLDKYEDVVQGLQKRGQQVVPLKYRRETPLKPIPVEALCDFEGEQGLISRGYSYTLQKNNGESWELMDSAGNKLIAPAVCFVIPPTDPEALALADSLGSQYRSVRQKAAGSKRTLQQRYEVLKTENPGDASDLQGRQLLAGLDKVASDLDRQEKAITGILRPPLEQGRAVQDSAERAKDLKNITNELLRIEPEKTRSTAEGEAFIQALPGSGTTPLLRTRVEDTNRKYEHLLQLLDLAQEKVDVANRLEKSLQQSWELLATHENHLNQDDTVPESSRVLDSKGQELAAMACELQAQKSLLGEVEQNLQAAKQCSSTLASRFQEHCPDLERQEAEVHKLGQRFNNLRQQVERRAQSLQSAKAAYEHFHRGHDHVLQFLVSIPSYEPQETDSLSQMETKLKNQKNLLDEIASREQEVQKICANSQQYQQAVKDYELEAEKLRSLLDLENGRRSHVSKRARLQSPATKVKEEEAALAAKFTEVYAINRQRLQNLEFALNLLRQQPEVEVTHETLQRNRPDSGVEEAWKIRKELDEETERRRQLENEVKSTQEEIWTLRNQGPQESVVRKEVLKKVPDPVLEESFQQLQRTLAEEQHKNQLLQEELEALQLQLRALEQETRDGGQEYVVKEVLRIEPDRAQADEVLQLREELEALRRQKGAREAEVLLLQQRVAALAEEKSRAQEKVTEKEVVKLQNDPQLEAEYQQLQEDHQRQDQLREKQEEELSFLQDKLKRLEKERAMAEGKITVKEVLKVEKDAATEREVSDLTRQYEDEAAKARASQREKTELLRKIWALEEENAKVVVQEKVREIVRPDPKAESEVANLRLELVEQERKYRGAEEQLRSYQSELEALRRRGPQVEVKEVTKEVIKYKTDPEMEKELQRLREEIVDKTRLIERCDLEIYQLKKEIQALKDTKPQVQTKEVVQEILQFQEDPQTKEEVASLRAKLSEEQKKQVDLERERASQEEQIARKEEELSRVKERVVQQEVVRYEEEPGLRAEASAFAESIDVELRQIDKLRAELRRLQRRRTELERQLEELERERQARREAEREVQRLQQRLAALEQEEAEAREKVTHTQKVVLQQDPQQAREHALLRLQLEEEQHRRQLLEGELETLRRKLAALEKAEVKEKVVLSESVQVEKGDTEQEIQRLKSSLEEESRSKRELDVEVSRLEARLSELEFHNSKSSKELDFLREENHKLQLERQNLQLETRRLQSEINMAATETRDLRNMTVADSGTNHDSRLWSLERELDDLKRLSKDKDLEIDELQKRLGSVAVKREQRENHLRRSIVVIHPDTGRELSPEEAHRAGLIDWNMFVKLRSQECDWEEISVKGPNGESSVIHDRKSGKKFSIEEALQSGRLTPAQYDRYVNKDMSIQELAVLVSGQK

SEQ ID NO: 42 - protein, 350 aa, Homo sapiens
MEPKRIREGYLVKKGSVFNTWKPMWVVLLEDGIEFYKKKSDNSPKGMIPLKGSTLTSPCQDFGKRMFVFKITTTKQQDHFFQAAFLEERDAWVRDIKKAIKCIEGGQKFARKSTRRSIRLPETIDLGALYLSMKDTEKGIKELNLEKDKKIFNHCFTGNCVIDWLVSNQSVRNRQEGLMIASSLLNEGYLQPAGDMSKSAVDGTAENPFLDNPDAFYYFPDSGFFCEENSSDDDVILKEEFRGVIIKQGCLLKQGHRRKNWKVRKFILREDPAYLHYYDPAGAEDPLGAIHLRGCVVTSVESNSNGRKSEEENLFEIITADEVHYFLQAATPKERTEWIRAIQMASRTGK

SEQ ID NO: 43 - protein, 140 aa, Homo sapiens
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 44 - protein, 505 aa, Homo sapiens
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 45 - protein, 92 aa, Homo sapiens
MTKLEEHLEGIVNIFHQYSVRKGHFDTLSKGELKQLLTKELANTIKNIKDKAVIDEIFQGLDANQDEQVDFQEFISLVAIALKAAHYHTHKE

SEQ ID NO: 46 - protein, 101 aa, Homo sapiens
MACPLEKALDVMVSTFHKYSGKEGDKFKLNKSELKELLTRELPSFLGKRTDEAAFQKLMSNLDSNRDNEVDFQEYCVFLSCIAMMCNEFFEGFPDKQPRKK

SEQ ID NO: 47 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 48 - protein, 201 aa, Homo sapiens
MTEKAPEPHVEEDDDDELDSKLNYKPPPQKSLKELQEMDKDDESLIKYKKTLLGDGPVVTDPKAPNVVVTRLTLVCESAPGPITMDLTGDLEALKKETIVLKEGSEYRVKIHFKVNRDIVSGLKYVQHTYRTGVKVDKATFMVGSYGPRPEEYEFLTPVEEAPKGMLARGTYHNKSFFTDDDKQDHLSWEWNLSIKKEWTE

SEQ ID NO: 49 - protein, 93 aa, Homo sapiens
MSGLRVYSTSVTGSREIKSQQSEVTRILDGKRIQYQLVDISQDNALRDEMRALAGNPKATPPQIVNGDQYCGDYELFVEAVEQNTLQEFLKLA

SEQ ID NO: 50 - protein, 288 aa, Homo sapiens [sequence omitted]

SEQ ID NO: 51 - protein, 2541 aa, Homo sapiens
MVALSLKISIGNVVKTMQFEPSTMVYDACRIIRERIPEAPAGPPSDFGLFLSDDDPKKGIWLEAGKALDYYMLRNGDTMEYRKKQRPLKIRMLDGTVKTIMVDDSKTVTDMLMTICARIGITNHDEYSLVRELMEEKKEEITGTLRKDKTLLRDEKKMEKLKQKLHTDDELNWLDHGRTLREQGVEEHETLLLRRKFFYSDQNVDSRDPVQLNLLYVQARDDILNGSHPVSFDKACEFAGFQCQIQFGPHNEQKHKAGFLDLKDFLPKEYVKQKGERKIFQAHKNCGQMSEIEAKVRYVKLARSLKTYGVSFFLVKEKMKGKNKLVPRLLGITKECVMRVDEKTKEVIQEWNLTNIKRWAASPKSFTLDFGDYQDGYYSVQTTEGEQIAQLIAGYIDIILKKKKSKDHFGLEGDEESTMLEDSVSPKKSTVLQQQYNRVGKVEHGSVALPAIMRSGASGPENFQVGSMPPAQQQITSGQMHRGHMPPLTSAQQALTGTINSSMQAVQAAQATLDDFDTLPPLGQDAASKAWRKNKMDESKHEIHSQVDAITAGTASVVNLTAGDPAETDYTAVGCAVTTISSNLTEMSRGVKLLAALLEDEGGSGRPLLQAAKGLAGAVSELLRSAQPASAEPRQNLLQAAGNVGQASGELLQQIGESDTDPHFQDALMQLAKAVASAAAALVLKAKSVAQRTEDSGLQTQVIAAATQCALSTSQLVACTKVVAPTISSPVCQEQLVEAGRLVAKAVEGCVSASQAATEDGQLLRGVGAAATAVTQALNELLQHVKAHATGAGPAGRYDQATDTILTVTENIFSSMGDAGEMVRQARILAQATSDLVNAIKADAEGESDLENSRKLLSAAKILADATAKMVEAAKGAAAHPDSEEQQQRLREAAEGLRMATNAAAQNAIKKKLVQRLEHAAKQAAASATQTIAAAQHAASTPKASAGPQPLLVQSCKAVAEQIPLLVQGVRGSQAQPDSPSAQLALIAASQSFLQPGGKMVAAAKASVPTIQDQASAMQLSQCAKNLGTALAELRTAAQKAQEACGPLEMDSALSVVQNLEKDLQEVKAAARDGKLKPLPGETMEKCTQDLGNSTKAVSSAIAQLLGEVAQGNENYAGIAARDVAGGLRSLAQAARGVAALTSDPAVQAIVLDTASDVLDKASSLIEEAKKAAGHPGDPESQQRLAQVAKAVTQALNRCVSCLPGQRDVDNALRAVGDASKRLLSDSLPPSTGTFQEAQSRLNEAAAGLNQAATELVQASRGTPQDLARASGRFGQDFSTFLEAGVEMAGQAPSQEDRAQVVSNLKGISMSSSKLLLAAKALSTDPAAPNLKSQLAAAARAVTDSINQLITMCTQQAPGQKECDNALRELETVRELLENPVQPINDMSYFGCLDSVMENSKVLGEAMTGISQNAKNGNLPEFGDAISTASKALCGFTEAAAQAAYLVGVSDPNSQAGQQGLVEPTQFARANQAIQMACQSLGEPGCTQAQVLSAATIVAKHTSALCNSCRLASARTTNPTAKRQFVQSAKEVANSTANLVKTIKALDGAFTEENRAQCRAATAPLLEAVDNLSAFASNPEFSSIPAQISPEGRAAMEPIVISAKTMLESAGGLIQTARALAVNPRDPPSWSVLAGHSRTVSDSIKKLITSMRDKAPGQLECETAIAALNSCLRDLDQASLAAVSQQLAPREGISQEALHTQMLTAVQEISHLIEPLANAARAEASQLGHKVSQMAQYFEPLTLAAVGAASKTLSHPQQMALLDQTKTLAESALQLLYTAKEAGGNPKQAAHTQEALEEAVQMMTEAVEDLTTTLNEAASAAGVVGGMVDSITQAINQLDEGPMGEPEGSFVDYQTTMVRTAKAIAVTVQEMVTKSNTSPEELGPLANQLTSDYGRLASEAKPAAVAAENEEIGSHIKHRVQELGHGCAALVTKAGALQCSPSDAYTKKELIECARRVSEKVSHVLAALQAGNRGTQACITAASAVSGIIADLDTTIMFATAGTLNREGTETFADHREGILKTAKVLVEDTKVLVQNAAGSQEKLAQAAQSSVATITRLADVVKLGAASLGAEDPETQVVLINAVKDVAKALGDLISATKAAAGKVGDDPAVWQLKNSAKVMVTNVTSLLKTVKAVEDEATKGTRALEATTEHIRQELAVFCSPEPPAKTSTPEDFIRMTKGITMATAKAVAAGNSCRQEDVIATANLSRRAIADMLRACKEAAYHPEVAPDVRLRALHYGRECANGYLELLDHVLLTLQKPSPELKQQLTGHSKRVAGSVTELIQAAEAMKGTEWVDPEDPTVIAENELLGAAAAIEAAAKKLEQLKPRAKPKEADESLNFEEQILEAAKSIAAATSALVKAASAAQRELVAQGKVGAIPANALDDGQWSQGLISAARMVAAATNNLCEAANAAVQGHASQEKLISSAKQVAASTAQLLVACKVKADQDSEAMKRLQAAGNAVKRASDNLVKAAQKAAAFEEQENETVVVKEKMVGGIAQIIAAQEEMLRKERELEEARKKLAQIRQQQYKFLPSELRDEH

SEQ ID NO: 52 - protein, 44 aa, Homo sapiens
MSDKPDMAEIEKFDKSKLKKTETQEKNPLPSKETIEQEKQAGES

SEQ ID NO: 53 - protein, 199 aa, Homo sapiens
MANRGPAYGLSREVQQKIEKQYDADLEQILIQWITTQCRKDVGRPQPGRENFQNWLKDGTVLCELINALYPEGQAPVKKIQASTMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 54 - protein, 285 aa, Homo sapiens
MMEAIKKKMQMLKLDKENALDRAEQAEAEQKQAEERSKQLEDELAAMQKKLKGTEDELDKYSEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEELDHALNDMTSI

SEQ ID NO: 55 - protein, 248 aa, Homo sapiens
MAGLNSLEAVKRKIQALQQQADEAEDRAQGLQRELDGERERREKAEGDVAALNRRIQLVEEELDRAQERLATALQKLEEAEKAADESERGMKVIENRAMKDEEKMEIQEMQLKEAKHIAEEADRKYEEVARKLVILEGELERAEERAEVSELKCGDLEEELKNVTNNLKSLEAASEKYSEKEDKYEEEIKLLSDKLKEAETRAEFAERTVAKLEKTIDDLEEKLAQAKEENVGLHQTLDQTLNELNCI

SEQ ID NO: 56 - protein, 512 aa, Homo sapiens
MGCIKSKGKDSLSDDGVDLKTQPVRNTERTIYVRDPTSNKQQRPVPESQLLPGQRFQTKDPEEQGDIVVALYPYDGIHPDDLSFKKGEKMKVLEEHGEWWKAKSLLTKKEGFIPSNYVAKLNTLETEEWFFKDITRKDAERQLLAPGNSAGAFLIRESETLKGSFSLSVRDFDPVHGDVIKHYKIRSLDNGGYYISPRITFPCISDMIKHYQKQADGLCRRLEKACISPKPQKPWDKDAWEIPRESIKLVKRLGAGQFGEVWMGYYNNSTKVAVKTLKPGTMSVQAFLEEANLMKTLQHDKLVRLYAVVTREEPIYIITEYMAKGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMTALSQGYRMPRVENCPDELYDIMKMCWKEKAEERPTFDYLQSVLDDFYTATEGQYQQQP

SEQ ID NO: 57 - protein, 380 aa, Homo sapiens
MSETVICSSRATVMLYDDGNKRWLPAGTGPQAFSRVQIYHNPTANSFRVVGRKMQPDQQVVINCAIVRGVKYNQATPNFHQWRDARQVWGLNFGSKEDAAQFAAGMASALEALEGGGPPPPPALPTWSVPNGPSPEEVEQQKRQQPGPSEHIERRVSNAGGPPAPPAGGPPPPPGPPPPPGPPPPPGLPPSGVPAAAHGAGGGPPPAPPLPAAQGPGGGGAGAPGLAAAIAGAKLRKVSKQEEASGGPTAPKAESGRSGGGGLMEEMNAMLARRRKATQVGEKTPKDESANQEEPEARVPAQSESVRRPWEKNSTTLPRMKSSSSVTTSETQPCTPSSSDYSDLQRVKQELLEEVKKELQKVKEEIIEAFVQELRKRGSP

SEQ ID NO: 58 - protein, 606 aa, Homo sapiens
MPYEIKKVFASLPQVERGVSKIIGGDPKGNNFLYTNGKCVILRNIDNPALADIYTEHAHQVVVAKYAPSGFYIASGDVSGKLRIWDTTQKEHLLKYEYQPFAGKIKDIAWTEDSKRIAVVGEGREKFGAVFLWDSGSSVGEITGHNKVINSVDIKQSRPYRLATGSDDNCAAFFEGPPFKFKFTIGDHSRFVNCVRFSPDGNRFATASADGQIYIYDGKTGEKVCALGGSKAHDGGIYAISWSPDSTHLLSASGDKTSKIWDVSVNSVVSTFPMGSTVLDQQLGCLWQKDHLLSVSLSGYINYLDRNNPSKPLHVIKGHSKSIQCLTVHKNGGKSYIYSGSHDGHINYWDSETGENDSFAGKGHTNQVSRMTVDESGQLISCSMDDTVRYTSLMLRDYSGQGVVKLDVQPKCVAVGPGGYAVVVCIGQIVLLKDQRKCFSIDNPGYEPEVVAVHPGGDTVAIGGVDGNVRLYSILGTTLKDEGKLLEAKGPVTDVAYSHDGAFLAVCDASKVVTVFSVADGYSENNVFYGHHAKIVCLAWSPDNEHFASGGMDMMVYVWTLSDPETRVKIQDAHRLHHVSSLAWLDEHTLVTTSHDASVKEWTITY

SEQ ID NO: 59 - protein, 572 aa, Homo sapiens
MAAPRPSPAISVSVSAPAFYAPQKKFGPVVAPKPKVNPFRPGDSEPPPAPGAQRAQMGRVGEIPPPPPEDFPLPPPPLAGDGDDAEGALGGAFPPPPPPIEESFPPAPLEEEIFPSPPPPPEEEGGPEAPIPPPPQPREKVSSIDLEIDSLSSLLDDMTKNDPFKARVSSGYVPPPVATPFSSKSSTKPAAGGTAPLPPWKSPSSSQPLPQVPAPAQSQTQFHVQPQPQPKPQVQLHVQSQTQPVSLANTQPRGPPASSPAPAPKFSPVTPKFTPVASKFSPGAPGGSGSQPNQKLGHPEALSAGTGSPQPPSFTYAQQREKPRVQEKQHPVPPPAQNQNQVRSPGAPGPLTLKEVEELEQLTQQLMQDMEHPQRQNVAVNELCGRCHQPLARAQPAVRALGQLFHIACFTCHQCAQQLQGQQFYSLEGAPYCEGCYTDTLEKCNTCGEPITDRMLRATGKAYHPHCFTCVVCARPLEGTSFIVDQANRPHCVPDYHKQYAPRCSVCSEPIMPEPGRDETVRVVALDKNFHMKCYKCEDCGKPLSIEADDNGCFPLDGHVLCRKCHTARAQT

SEQ ID NO: 60 - protein, 7 aa, synthetic construct
SLESALK

SEQ ID NO: 61 - protein, 7 aa, synthetic construct
AVEHINK

SEQ ID NO: 62 - protein, 11 aa, synthetic construct
ASLGTGTASPR

SEQ ID NO: 63 - protein, 28 aa, synthetic construct
  MOD_RES 15: note = PHOSPHORYLATION
  MOD_RES 23: note = PHOSPHORYLATION
ATASPRPSSGNIPSSPTASGGGSPTSPR

SEQ ID NO: 64 - protein, 9 aa, synthetic construct
  MOD_RES 3: note = PHOSPHORYLATION
GSSLKIEER

SEQ ID NO: 65 - protein, 8 aa, synthetic construct
GIQNDLTK

SEQ ID NO: 66 - protein, 12 aa, synthetic construct
NSNPALNDNLEK

SEQ ID NO: 67 - protein, 11 aa, synthetic construct
  MOD_RES 3: note = PHOSPHORYLATION
RPTATGDYDKK

SEQ ID NO: 68 - protein, 21 aa, synthetic construct
LYGSAGPPPTGEEDTAEKDEL

SEQ ID NO: 69 - protein, 9 aa, synthetic construct
AEISFEDRK

SEQ ID NO: 70 - protein, 9 aa, synthetic construct
SSFTVDCSK

SEQ ID NO: 71 - protein, 9 aa, synthetic construct
TPCEEILVK

SEQ ID NO: 72 - protein, 10 aa, synthetic construct
  MOD_RES 8: note = PHOSPHORYLATION
KVIYSQPSAR

SEQ ID NO: 73 - protein, 15 aa, synthetic construct
  MOD_RES 10: note = PHOSPHORYLATION
NSASATTPLSGNSSR

SEQ ID NO: 74 - protein, 9 aa, synthetic construct
TQDQISNIK

SEQ ID NO: 75 - protein, 10 aa, synthetic construct
  MOD_RES 1: note = PHOSPHORYLATION
SPQPLGGSHR

SEQ ID NO: 76 - protein, 11 aa, synthetic construct
AQQELEEQTRR

SEQ ID NO: 77 - protein, 7 aa, synthetic construct
SDEMNVK

SEQ ID NO: 78 - protein, 12 aa, synthetic construct
GCTDNLTLTVAR

SEQ ID NO: 79 - protein, 8 aa, synthetic construct
  MOD_RES 4: note = PHOSPHORYLATION
HPPSPPLR

SEQ ID NO: 80 - protein, 14 aa, synthetic construct
GSTLTSPCQDFGKR

SEQ ID NO: 81 - protein, 11 aa, synthetic construct
EGVHGGLINKK

SEQ ID NO: 82 - protein, 14 aa, synthetic construct
STGGAPTFNVTVTK

SEQ ID NO: 83 - protein, 15 aa, synthetic construct
  MOD_RES 2: note = PHOSPHORYLATION
KTGSYGALAEITASK

SEQ ID NO: 84 - protein, 11 aa, synthetic construct
ANHEEVLAAGK

SEQ ID NO: 85 - protein, 19 aa, synthetic construct
IQYQLVDISQDNALRDEMR

SEQ ID NO: 86 - protein, 10 aa, synthetic construct
VYSTSVTGSR

SEQ ID NO: 87 - protein, 18 aa, synthetic construct
  MOD_RES 5: note = PHOSPHORYLATION
  MOD_RES 9: note = PHOSPHORYLATION
AKTQTPPVSPAPQPTEER

SEQ ID NO: 88 - protein, 11 aa, synthetic construct
ALDGAFTEENR

SEQ ID NO: 89 - protein, 8 aa, synthetic construct
TIMVDDSK

SEQ ID NO: 90 - protein, 8 aa, synthetic construct
GPAYGLSR

SEQ ID NO: 91 - protein, 7 aa, synthetic construct
NMACVQR

SEQ ID NO: 92 - protein, 16 aa, synthetic construct
AQKDEEKMEIQEIQLK

SEQ ID NO: 93 - protein, 6 aa, synthetic construct
AEFAER

SEQ ID NO: 94 - protein, 7 aa, synthetic construct
LATALQK

SEQ ID NO: 95 - protein, 7 aa, synthetic construct
AEVSELK

SEQ ID NO: 96 - protein, 12 aa, synthetic construct
EKAEGDVAALNR

SEQ ID NO: 97 - protein, 15 aa, synthetic construct
KIQALQQQADEAEDR

SEQ ID NO: 98 - protein, 8 aa, synthetic construct
SLEAASEK

SEQ ID NO: 99 - protein, 8 aa, synthetic construct
TIDDLEEK

SEQ ID NO: 100 - protein, 9 aa, synthetic construct
NQPQFQQMR

SEQ ID NO: 101 - protein, 14 aa, synthetic construct
LYSILGTTLKDEGK

SEQ ID NO: 102 - protein, 8 aa, synthetic construct
VINSVDIK

SEQ ID NO: 103 - protein, 9 aa, synthetic construct
YEYQPFAGK

SEQ ID NO: 104 - protein, 19 aa, synthetic construct
  MOD_RES 14: note = PHOSPHORYLATION
GPPASSPAPAPKFSPVTPK

SEQ ID NO: 105 - protein, 11 aa, synthetic construct
  MOD_RES 3: note = PHOSPHORYLATION
RPTATGDYDKK

SEQ ID NO: 106 - protein, 15 aa, synthetic construct
  MOD_RES 2: note = PHOSPHORYLATION
KTGSYGALAEITASK

SEQ ID NO: 107 - protein, 10 aa, synthetic construct
  MOD_RES 1: note = PHOSPHORYLATION
SPQPLGGSHR

SEQ ID NO: 108 - protein, 7 aa, synthetic construct
  MOD_RES 4: note = PHOSPHORYLATION
KVDSLKK

SEQ ID NO: 109 - protein, 6 aa, synthetic construct
  MOD_RES 5: note = PHOSPHORYLATION
YSGHSL

SEQ ID NO: 110 - protein, 11 aa, synthetic construct
  MOD_RES 2: note = PHOSPHORYLATION
LSLTDPLVAER

SEQ ID NO: 111 - protein, 7 aa, synthetic construct
  MOD_RES 4: note = PHOSPHORYLATION
VKESITR

SEQ ID NO: 112 - protein, 11 aa, synthetic construct
  MOD_RES 3: note = PHOSPHORYLATION
NFSDNQLQEGK

SEQ ID NO: 113 - protein, 9 aa, synthetic construct
  MOD_RES 3: note = PHOSPHORYLATION
GSSLKIEER

SEQ ID NO: 114 - protein, 17 aa, synthetic construct
  MOD_RES 5: note = PHOSPHORYLATION
EMLASDDEEDVSSKVEK

SEQ ID NO: 115 - protein, 18 aa, synthetic construct
  MOD_RES 5: note = PHOSPHORYLATION
  MOD_RES 9: note = PHOSPHORYLATION
AKTQTPPVSPAPQPTEER

SEQ ID NO: 116 - protein, 11 aa, synthetic construct
  MOD_RES 3: note = PHOSPHORYLATION
YGSQEHPIGDK

SEQ ID NO: 117 - protein, 12 aa, synthetic construct
  MOD_RES 11: note = PHOSPHORYLATION
AYSSFGGGRGSR

SEQ ID NO: 118 - protein, 6 aa, synthetic construct
  MOD_RES 4: note = PHOSPHORYLATION
IRESPK

SEQ ID NO: 119 - protein, 19 aa, synthetic construct
  MOD_RES 14: note = PHOSPHORYLATION
GPPASSPAPAPKFSPVTPK

SEQ ID NO: 120 - protein, 11 aa, synthetic construct
  MOD_RES 1: note = PHOSPHORYLATION
SPGAPGPLTLK

SEQ ID NO: 121 - protein, 8 aa, synthetic construct
  MOD_RES 4: note = PHOSPHORYLATION
HPPSPPLR

SEQ ID NO: 122 - protein, 14 aa, synthetic construct
  MOD_RES 3: note = PHOSPHORYLATION
RDSLDKSGLFPEWK

SEQ ID NO: 123 - protein, 28 aa, synthetic construct
  MOD_RES 9: note = PHOSPHORYLATION
  MOD_RES 23: note = PHOSPHORYLATION
ATASPRPSSGNIPSSPTASGGGSPTSPR

SEQ ID NO: 124 - protein, 15 aa, synthetic construct
  MOD_RES 10: note = PHOSPHORYLATION
NSASATTPLSGNSSR

SEQ ID NO: 125 - protein, 10 aa, synthetic construct
  MOD_RES 8: note = PHOSPHORYLATION
KVIYSQPSAR

SEQ ID NO: 126 - protein, 14 aa, synthetic construct
AEAGVPAEFSIWTR

SEQ ID NO: 127 - protein, 8 aa, synthetic construct
AEGPGLSR

SEQ ID NO: 128 - protein, 7 aa, synthetic construct
  MOD_RES 4: note = PHOSPHORYLATION
VKESITR

SEQ ID NO: 129 - protein, 9 aa, synthetic construct
AEISFEDRK

SEQ ID NO: 130 - protein, 16 aa, synthetic construct
AFGPGLQGGSAGSPAR

SEQ ID NO: 131 - protein, 8 aa, synthetic construct
AGGPGLER

SEQ ID NO: 132 - protein, 17 aa, synthetic construct
AGQSAAGAAPGGGVDTR

SEQ ID NO: 133 - protein, 9 aa, synthetic construct
AGVAPLQVK

SEQ ID NO: 134 - protein, 13 aa, synthetic construct
ANLPQSFQVDTSK

SEQ ID NO: 135 - protein, 24 aa, synthetic construct
ASGPGLNTTGVPASLPVEFTIDAK

SEQ ID NO: 136 - protein, 19 aa, synthetic construct
ATCAPQHGAPGPGPADASK

SEQ ID NO: 137 - protein, 13 aa, synthetic construct
AWGPGLEGGVVGK

SEQ ID NO: 138 - protein, 16 aa, synthetic construct
  MOD_RES 3: note = PHOSPHORYLATION
APSVANVGSHCDLSLK

SEQ ID NO: 139 - protein, 14 aa, synthetic construct
AYGPGIEPTGNMVK

SEQ ID NO: 140 - protein, 8 aa, synthetic construct
CSGPGLER

SEQ ID NO: 141 - protein, 12 aa, synthetic construct
CSGPGLSPGMVR

SEQ ID NO: 142 - protein, 16 aa, synthetic construct
DAGEGGLSLAIEGPSK

SEQ ID NO: 143 - protein, 19 aa, synthetic construct
DAGEGLLAVQITDPEGKPK

SEQ ID NO: 144 - protein, 10 aa, synthetic construct
DAPQDFHPDR

SEQ ID NO: 145 - protein, 12 aa, synthetic construct
  MOD_RES 7: note = PHOSPHORYLATION
CSGPGLSPGMVR

SEQ ID NO: 146 - protein, 21 aa, synthetic construct
DGSCGVAYVVQEPGDYEVSVK

SEQ ID NO: 147 - protein, 9 aa, synthetic construct
DLAEDAPWK

SEQ ID NO: 148 - protein, 15 aa, synthetic construct
DVDIIDHHDNTYTVK

SEQ ID NO: 149 - protein, 16 aa, synthetic construct
EAGAGGLAIAVEGPSK

SEQ ID NO: 150 - protein, 8 aa, synthetic construct
EEPCLLKR

SEQ ID NO: 151 - protein, 17 aa, synthetic construct
EGPYSISVLYGDEEVPR

SEQ ID NO: 152 - protein, 10 aa, synthetic construct
ENGVYLIDVK

SEQ ID NO: 153 - protein, 10 aa, synthetic construct
ETGEHLVHVK

SEQ ID NO: 154 - protein, 23 aa, synthetic construct
FNEEHIPDSPFVVPVASPSGDAR

SEQ ID NO: 155 - protein, 6 aa, synthetic construct
FTVETR

SEQ ID NO: 156 - protein, 18 aa, synthetic construct
GAGTGGLGLAVEGPSEAK

SEQ ID NO: 157 - protein, 24 aa, synthetic construct
GLVEPVDVVDNADGTQTVNYVPSR

SEQ ID NO: 158 - protein, 10 aa, synthetic construct
GTVEPQLEAR

SEQ ID NO: 159 - protein, 14 aa, synthetic construct
IECDDKGDGSCDVR

SEQ ID NO: 160 - protein, 19 aa, synthetic construct
  MOD_RES 14: note = PHOSPHORYLATION
  MOD_RES 15: note = PHOSPHORYLATION
IPEISIQDMTAQVTSPSGK

SEQ ID NO: 161 - protein, 12 aa, synthetic construct
IVGPSGAAVPCK

SEQ ID NO: 162 - protein, 9 aa, synthetic construct
KGEITGEVR

SEQ ID NO: 163 - protein, 26 aa, synthetic construct
LQVEPAVDTSGVQCYGPGIEGQGVFR

SEQ ID NO: 164 - protein, 11 aa, synthetic construct
MDCQECPEGYR

SEQ ID NO: 165 - protein, 7 aa, synthetic construct
MSCMDNK

SEQ ID NO: 166 - protein, 17 aa, synthetic construct
  MOD_RES 4: note = PHOSPHORYLATION
RAPSVANVGSHCDLSLK

SEQ ID NO: 167 - protein, 22 aa, synthetic construct
SAGQGEVLVYVEDPAGHQEEAK

SEQ ID NO: 168 - protein, 9 aa, synthetic construct
SPFEVYVDK

SEQ ID NO: 169 - protein, 15 aa, synthetic construct
SPFSVAVSPSLDLSK

SEQ ID NO: 170 - protein, 17 aa, synthetic construct
SPYTVTVGQACNPSACR

SEQ ID NO: 171 - protein, 9 aa, synthetic construct
SSFTVDCSK

SEQ ID NO: 172 - protein, 15 aa, synthetic construct
TFSVWYVPEVTGTHK

SEQ ID NO: 173 - protein, 16 aa, synthetic construct
TGVAVNKPAEFTVDAK

SEQ ID NO: 174 - protein, 17 aa, synthetic construct
THEAEIVEGENHTYCIR

SEQ ID NO: 175 - protein, 9 aa, synthetic construct
TPCEEILVK

SEQ ID NO: 176 - protein, 16 aa, synthetic construct
VANPSGNLTETYVQDR

SEQ ID NO: 177 - protein, 17 aa, synthetic construct
VAQPTITDNKDGTVTVR

SEQ ID NO: 178 - protein, 12 aa, synthetic construct
VDVGKDQEFTVK

SEQ ID NO: 179 - protein, 13 aa, synthetic construct
VEPGLGADNSVVR

SEQ ID NO: 180 - protein, 16 aa, synthetic construct
VQVQDNEGCPVEALVK

SEQ ID NO: 181 - protein, 17 aa, synthetic construct
VTAQGPGLEPSGNIANK

SEQ ID NO: 182 - protein, 12 aa, synthetic construct
VTVLFAGQHIAK

SEQ ID NO: 183 - protein, 12 aa, synthetic construct
WGDEHIPGSPYR

SEQ ID NO: 184 - protein, 12 aa, synthetic construct
YGGDEIPFSPYR

SEQ ID NO: 185 - protein, 22 aa, synthetic construct
YTPVQQGPVGVNVTYGGDPIPK

SEQ ID NO: 186 - protein, 13 aa, synthetic construct
  MOD_RES 3: note = PHOSPHORYLATION
RLTVSSLQESGLK